(19) United States
(12) Patent Application Publication
(10) Pub. No.: US 2010/0152434 A1
(43) Pub. Date: Jun. 17, 2010

(54) Protein Kinase-binding Nucleosides and Associated Methods

(76) Inventor: Matt A. Peterson, Provo, UT (US)

Correspondence Address: THORPE NORTH & WESTERN, LLP., P.O. Box 1219, SANDY, UT, 84091-1219, US

(21) Appl. No.: 12/627,898

(22) Filed: Nov. 30, 2009

Related U.S. Application Data
(63) Continuation-in-part of application No. PCT/US08/65334, filed on May 30, 2008.
(60) Provisional application No. 60/932,528, filed on May 30, 2007.

(51) Int. Cl. C07H 19/16

(52) U.S. Cl. 536/27.22; 536/27.23

(57) ABSTRACT

Therapeutically active nucleosides and associated methods are provided. In one aspect, a nucleoside molecule having a general structural similar to ATP. Such nucleosides have a structure that allows binding to, and subsequent regulation of, protein kinase molecules. As such, the nucleosides of the present invention have may be capable of treating a variety of kinase-related medical disorders.

### PRIORITY DATA

This application is a continuation in-part of PCT Application No. PCT/U.S.08/65334, filed on May 30, 2008, which claims the benefit of U.S. Provisional Patent Application Ser. No. 60/932,528, filed on May 30, 2007, both of which are incorporated herein by reference in their entirety.

### FIELD OF THE INVENTION

The present invention relates to novel nucleosides having therapeutic activity. Accordingly, this invention involves the fields of chemistry, medicine and other health sciences.

### BACKGROUND OF THE INVENTION

Protein kinase molecules are enzymes that modify other proteins through the addition of phosphate groups in a process known as phosphorylation. Phosphorylation generally results in a functional change of the target protein through modification of enzymatic activity, protein-protein interactions, etc. Kinases are known to regulate many cellular pathways, particularly those involved in signal transduction. In some cases phosphorylation occurs through the removal of a phosphate group from Adenosine Triphosphate (ATP) and its subsequent covalent attachment to one of three amino acids that have a free hydroxyl group. Most kinases act on both serine and threonine, while others act on tyrosine, and a number (dual specificity kinases) act on all three.

Because protein kinases can have a profound effect on cells, the activity of these molecules in physiological systems tend to be highly regulated. Kinases can be turned on or off by phosphorylation, by binding of activator proteins or inhibitor proteins, by binding of small molecules, or by controlling their location in the cell relative to their substrates.

Deregulated kinase activity is a frequent cause of disease, particularly cancer, where kinases regulate many aspects that control cell growth, cell movement, and cell death. Accordingly, pharmaceutical agents that reduce or otherwise limit such deregulated kinase activity may be beneficial in the treatment of kinase related conditions such as cancer.

### BRIEF DESCRIPTION OF THE DRAWINGS

FIG. 1 shows a diagram of ATP in the ATP binding site of a protein kinase molecule according to one aspect of the present invention.

FIG. 2 shows a diagram of a nucleoside in the ATP binding site of a protein kinase molecule according to another aspect of the present invention.

FIG. 3 shows a series of chemical reaction schemes describing the generation of various compounds according to yet another aspect of the present invention.

FIG. 4 shows a series of chemical reaction schemes describing the generation of various compounds according to a further aspect of the present invention.

FIG. 5 shows a series of chemical reaction schemes describing the generation of various compounds according to yet a further aspect of the present invention.

FIG. 6 shows a series of chemical reaction schemes describing the generation of various compounds according to another aspect of the present invention.

FIG. 7 shows a series of chemical reaction schemes describing the generation of various compounds according to yet another aspect of the present invention.

FIG. 8 shows a series of chemical reaction schemes describing the generation of various compounds according to a further aspect of the present invention.

FIG. 9 shows a series of chemical reaction schemes describing the generation of various compounds according to yet a further aspect of the present invention.

FIG. 10 shows a series of chemical reaction schemes describing the generation of various compounds according to another aspect of the present invention.

FIG. 11 shows a series of chemical reaction schemes describing the generation of various compounds according to yet another aspect of the present invention.

### DEFINITIONS OF KEY TERMS

In describing and claiming the present invention, the following terminology will be used in accordance with the definitions set forth below.

The singular forms “a,” “an,” and, “the” include plural referents unless the context clearly dictates otherwise. Thus, for example, reference to “a molecule” includes reference to one or more of such molecules, reference to “a Compound” includes reference to one or more such Compounds, and reference to “an antibody” includes reference to one or more of such antibodies.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, the terms “molecule” and “compound” may be used interchangeably.

As used herein, the terms “formulation” and “composition” are used interchangeably and refer to a mixture of two or more compounds, elements, or molecules. In some aspects the terms “formulation” and “composition may be used to refer to a mixture of a nucleoside with a carrier or other excipients.

“Administration,” and “administering” refer to the manner in which an active agent is presented to a subject. Administration can be accomplished by various art-known routes such as oral, parenteral, transdermal, inhalation, implantation, etc. Thus, an oral administration can be achieved by swallowing, chewing, sucking of an oral dosage form comprising the drug. Parenteral administration can be achieved by injecting a drug composition intravenously, intra-arterially, intramuscularly, intrathecally, or subcutaneously, etc. Transdermal administration can be accomplished by applying, pasting, rolling, attaching, pouring, pressing, rubbing, etc., of a transdermal preparation onto a skin surface. These and additional methods of administration are well-known in the art.

As used herein, “effective amount” of an enhancer refers to an amount sufficient to increase the penetration of a drug through the skin to a selected degree. Methods for assaying the characteristics of permeation enhancers are well-known in the art. See, for example, Merritt et al., “Diffusion Apparatus for Skin Penetration,” J. of Controlled Release 61 (1984), incorporated herein by reference in its entirety. Thus, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier,” and “carrier” may be used interchangeably, and refer to any inert and pharmaceutically acceptable material that has substantially no biological activity, and makes up a substantial part of the formulation. The carrier may be polymeric, such as an adhesive, or non-polymeric and is generally admixed with other components of the composition (e.g., drug, binders, fillers, penetration enhancers, anti-irritants, emollients, lubricants, etc., as needed) to comprise the formulation.

As used herein, “excipient” refers to substantially inert substance which may be combined with an active agent and a carrier to achieve a specific dosage formulation for delivery to a subject, or to provide a dosage form with specific performance properties. For example, excipients may include binders, lubricants, etc., but specifically exclude active agents and carriers.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### DETAILED DESCRIPTION

It has now been discovered that nucleoside compounds having a general structure as described herein bind to various protein kinases. As was described above, protein kinase deregulation can result in numerous conditions, including cancer. As such, regulation of protein kinases according to aspects of the present invention may prove important in the treatments of numerous conditions and disorders, including cancers.

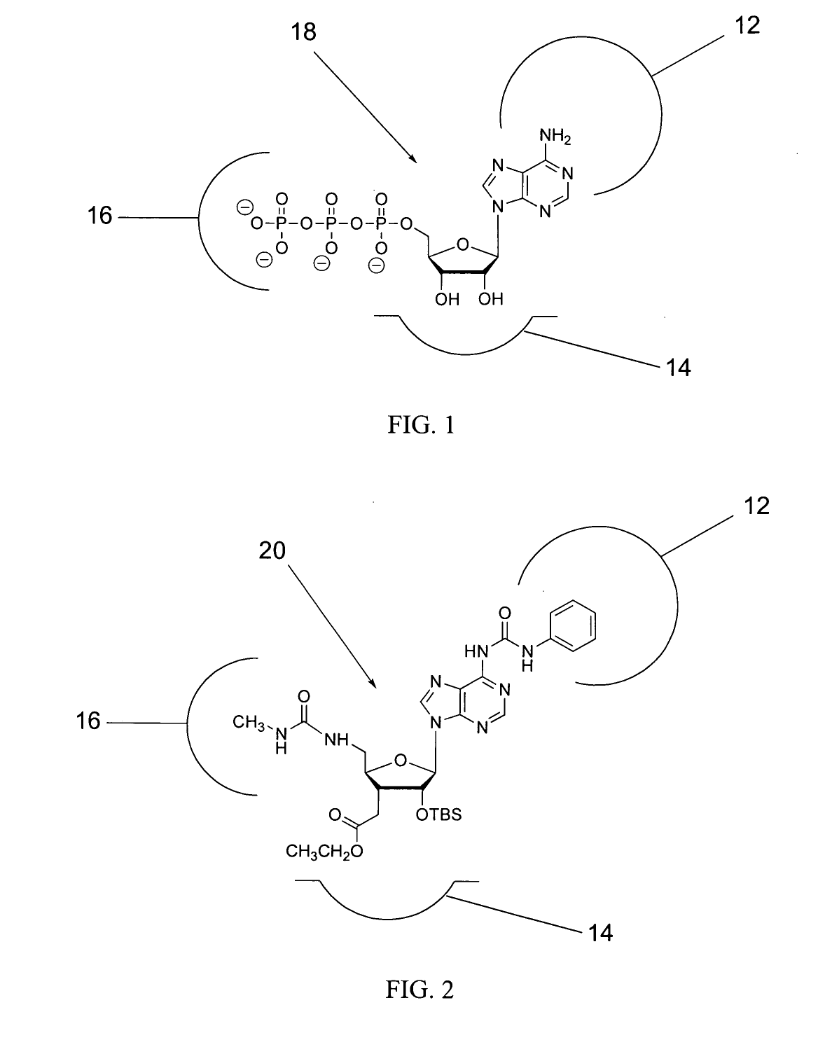

The nucleoside structure of the present invention have a structural similarity to adenosine 5′-triphosphate (ATP), and thus may bind in the ATP binding site of a protein kinase to exert anticancer functionality. It is believed that ATP binds in the ATP binding site of a protein kinase within a cleft formed between two lobes of the kinase molecule in an orientation as shown in FIG. 1. The ATP binding site includes, inter alia, a hydrophobic pocket 12, a sugar binding pocket 14, and a triphosphate binding pocket 16. An ATP molecule 18 is shown in the ATP binding site of the protein kinase. It appears that the hydrophobic pocket 12 is not utilized by ATP, but may be exploited by many kinase inhibitors. The hydrophobic pocket may play a role in inhibitor selectivity.

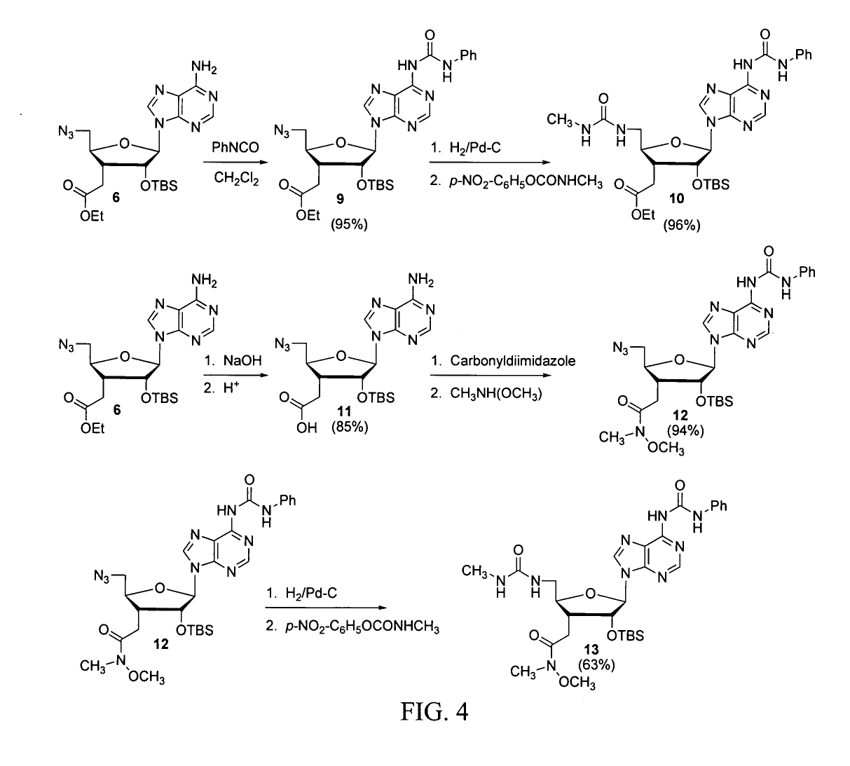

As is shown in FIG. 2, a representative example structure 20 (Compound 10, FIG. 4) fits into the ATP binding site in a similar orientation as compared to the ATP molecule. Compound 10 has now been shown to have an affinity for binding in the ATP binding site, as is shown below, and therefore is a good candidate for a nucleoside having anticancer activity. Furthermore, Compound 10 has now been shown to inhibit growth of various cancer cell lines, as is also shown below.

Once having an understanding of the binding of Compound 10 to the ATP binding site of a protein kinase molecule, one of ordinary skill in the art would appreciate that a variety of modifications to the structure of Compound 10 and related molecules would result in nucleosides having the same if not improved binding affinity for the ATP binding site. For example, by modifying a sidegroup of the nucleoside to reduce steric hindrance with the kinase can improve the binding affinity of the nucleoside to the binding site. Numerous molecules are thus contemplated, and it should be noted that any nucleoside having the general structure demonstrated herein would be considered to be within the present scope.

Aspects of the present invention provide novel nucleoside molecules and methods for their making and use. In one aspect of the present invention, for example, a molecule is provided having the structure as in Compound 1:

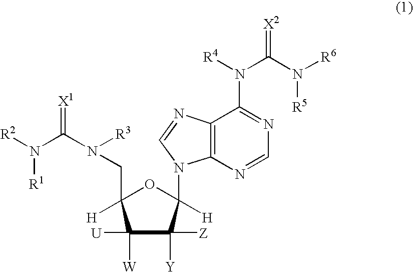

In such molecules, R¹, R², R⁵, and R⁶, can be selected independently from H, HO—, CH₃O—, CH₃—, HOCH₂CH₂—, HOCH₂CH₂OCH₂CH₂—, NH₂CH₂CH₂—, R⁷NHCH₂CH₂—, (R⁷)₂NCH₂CH₂—, NH₂CH₂CH₂NHCH₂CH₂—, R⁷NHCH₂CH₂NHCH₂CH₂—, (R⁷)₂NCH₂CH₂NHCH₂CH₂—, R⁸CO—, a mono-, di-, or tri-cyclic aryl from C₆ to C₁₄, a mono-, di-, or tri-cyclic aryl from C₆ to C₁₄ mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂. Additionally, R⁷ can be an alkyl from C₁ to C₅, R⁸ can be H₂N—, HOHN—, alkyl from C₁ to C₁₀, alkenyl from C₂ to C₁₀, or phenyl, R⁹ can be alkyl from C₁ to C₂₀, and R³ and R⁴ can include members selected independently from H, HO—, CH₃—, or CH₃CH₂—. Furthermore, X¹ and X² can include members selected independently from O and S, U can include a member selected from H, HO—, F, CF₃—, and W can include a member selected from H, HO—, F, CF₃—, CH₃CH₂O₂CCH₂—, CH₃(CH₃O)NCOCH₂—, HOCH₂CH₂O—, NH₂COCH₂—, CH₃NHCOCH₂—, (CH₃)₂NCOCH₂—, HOCH₂CH₂NHCOCH₂—, HSCH₂CH₂NHCOCH₂—, R⁹O—, and an O-trialkylsilyl containing three to sixteen carbons. Also, Y can include a member selected from H, HO—, F, CF₃—, HOCH₂CH₂O—, R⁹O—, and an O-trialkylsilyl containing three to sixteen carbons, and Z can include a member selected from H, F, HO—, CF₃—, and R⁹O—.

In a more specific aspect of Compound 1, a molecule is provided having the structure as in Compound 8:

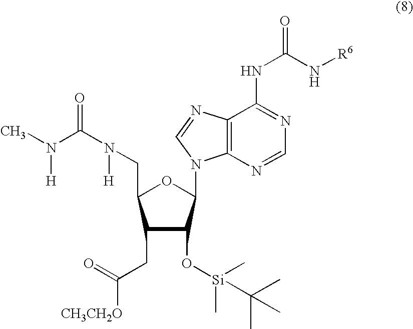

Such a molecule is essentially Compound 1 where R¹ is H, R² is CH₃, R³ is H, R⁴ is H, R⁵ is H, U is H, W is CH₃CH₂CCH₂—, Z is H, Y is O-tert-butyldimethylsilyl, X¹ is O, and X² is O. Additionally, R⁶ can be a group including a mono-, di-, or tri-cyclic aryl from C₆ to C₁₄, a mono-, di-, or tri-cyclic aryl from C₆ to C₁₄ mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂, where R⁹ is alkyl from C₁ to C₁₂.

In another more specific aspect of Compound 8, a molecule is provided having the structure as in Compound 10, where R⁶ is phenyl:

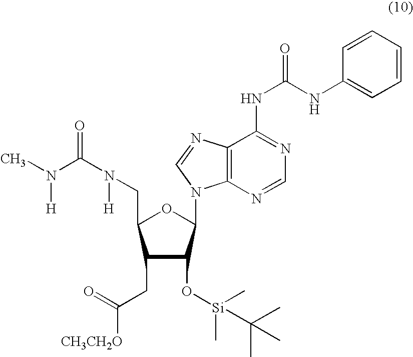

Numerous additional nucleosides having the general structure of Compound 8 are additionally contemplated. For example, in one aspect R⁶ can be a group including a mono-, di-, or tri-cyclic aryl from C₆ to C₁₄. In another aspect, R⁶ can be a group including a mono-, di-, or tri-cyclic aryl from C₆ to C₁₄ mono-, di-, tri-, or poly-substituted with a member selected independently from F, Cl, Br, or I. In yet another aspect, R⁶ can be a group including a mono-, di-, or tri-cyclic aryl from C₆ to C₁₄ mono-, di-, tri-, or poly-substituted with alkoxy (R⁹O—), where R⁹ is alkyl from C₁ to C₁₂. In a further aspect, R⁶ can be a group including a mono-, di-, or tri-cyclic aryl from C₆ to C₁₄ mono-, di-, tri-, or poly-substituted with nitro (NO₂), nitroso (NO), or azido (N₃). In yet a further aspect, R⁶ can be a group including a mono-, di-, or tri-cyclic aryl from C₆ to C₁₄ mono-, di-, tri-, or poly-substituted with alkyl from C₂ to C₁₂, alkenyl from C₂ to C₁₂, alkynyl from C₂ to C₁₂, or acyl from C₂ to C₁₂. In another aspect, R⁶ can be a group including an O, N, or S mono- or bi-cyclic heterocycle having from two to nine carbon atoms. In yet another aspect, R⁶ can be an O, N, or S mono- or bi-cyclic heterocycle having from two to nine carbon atoms and mono-, di-, tri-, or poly-substituted with a group including F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to Cₚ, and where R⁹ is alkyl from C₁ to C₁₂.

In another more specific aspect of Compound 1, a molecule is provided having the structure as in Compound 13:

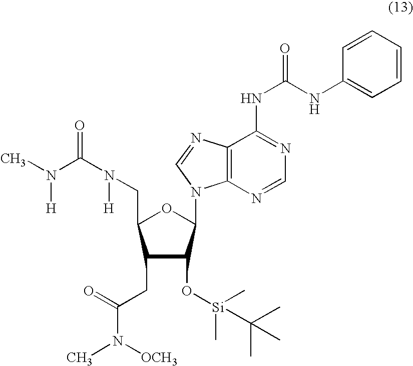

Such a molecule is essentially Compound 1 where R¹ is H, R² is CH₃, R³ is H, R⁴ is H, R⁵ is H, U is H, W is CH₃(CH₃O)NCOCH₂—, Z is H, Y is O-tert-butyldimethylsilyl, X¹ is O, X² is O, and R⁶ is phenyl.

In another more specific aspect of Compound 1, a molecule is provided having the structure as in Compound 17:

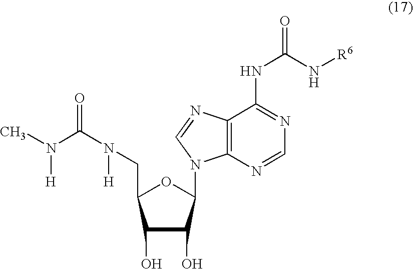

Such a molecule is essentially Compound 1 where R¹ is H, R² is CH₃, R³ is H, R⁴ is H, R⁵ is H, U is H, W is OH, Z is H, Y is OH, X¹ is O, X² is O. Additionally, R⁶ is a member selected from a mono-, di-, or tri-cyclic aryl from C₆ to C₁₄, a mono-, di-, or tri-cyclic aryl from C₆ to C₁₄ mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂. Additionally, R⁹ can be alkyl from C₁ to C₁₂.

In another more specific aspect of Compound 17, a molecule is provided having the structure as in Compound 23, where R⁶ is phenyl:

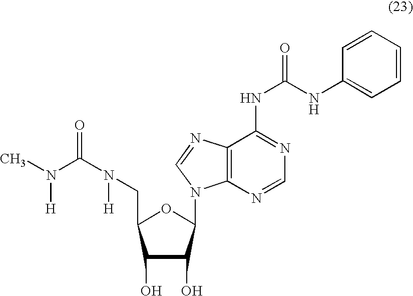

Numerous additional nucleosides having the general structure of Compound 17 are additionally contemplated. For example, in one aspect R⁶ can be a mono-, di-, or tri-cyclic aryl from C₆ to C₁₄. In another aspect, R⁶ can be a mono-, di-, or tri-cyclic aryl from C₆ to C₁₄ mono-, di-, tri-, or poly-substituted with a member selected independently from F, Cl, Br, or I. In yet another aspect, R⁶ can be a mono-, di-, or tri-cyclic aryl from C₆ to C₁₄ mono-, di-, tri-, or poly-substituted with alkoxy (R⁹O—), where R⁹ is alkyl from C₁ to C₁₂. In a further aspect, R⁶ can be a mono-, di-, or tri-cyclic aryl from C₆ to C₁₄ mono-, di-, tri-, or poly-substituted with nitro (NO₂), nitroso (NO), or azido (N₃). In yet a further aspect, R⁶ can be a mono-, di-, or tri-cyclic aryl from C₆ to C₁₄ mono-, di-, tri-, or poly-substituted with alkyl from C₂ to C₁₂, alkenyl from C₂ to C₁₂, alkynyl from C₂ to C₁₂, or acyl from C₂ to C₁₂. In another aspect, R⁶ can be an O, N, or S mono- or bi-cyclic heterocycle having from two to nine carbon atoms. In yet another aspect, R⁶ is an O, N, or S mono- or bi-cyclic heterocycle having from two to nine carbon atoms and mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂, where R⁹ is alkyl from C₁ to C₁₂.

In yet another more specific aspect of Compound 1, a molecule is provided having the structure as in Compound 16:

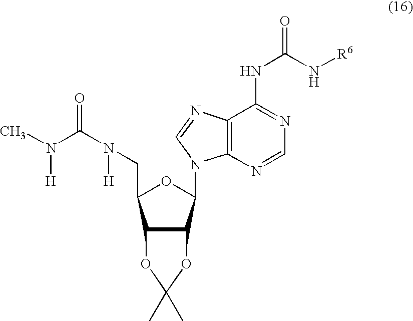

Such a molecule is essentially Compound 1 where R¹ is H, R² is CH₃, R³ is H, R⁴ is H, R⁵ is H, U is H, Z is H, W and Y are —OC(CH₃)₂O—, X¹ is O, X² is O. Additionally, R⁶ is a member selected from a mono-, di-, or tri-cyclic aryl from C₆ to C₁₄, a mono-, di-, or tri-cyclic aryl from C₆ to C₁₄ mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂, and where R⁹ is alkyl from C₁ to C₁₂.

In another more specific aspect of Compound 16, a molecule is provided having the structure as in Compound 22, where R⁶ is phenyl:

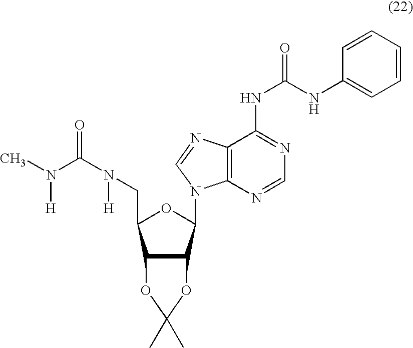

Numerous additional nucleosides having the general structure of Compound 16 are additionally contemplated. For example, in one aspect R⁶ can be a mono-, di-, or tri-cyclic aryl from C₆ to C₁₄. In another aspect, R⁶ can be a mono-, di-, or tri-cyclic aryl from C₆ to C₁₄ mono-, di-, tri-, or poly-substituted with a member selected independently from F, Cl, Br, or I. In yet another aspect, R⁶ can be a mono-, di-, or tri-cyclic aryl from C₆ to C₁₄ mono-, di-, tri-, or poly-substituted with alkoxy (R⁹O—), where R⁹ is alkyl from C₁ to C₁₂. In a further aspect, R⁶ can be a mono-, di-, or tri-cyclic aryl from C₆ to C₁₄ mono-, di-, tri-, or poly-substituted with nitro (NO₂), nitroso (NO), or azido (N₃). In yet a further aspect, R⁶ can be a mono-, di-, or tri-cyclic aryl from C₆ to C₁₄ mono-, di-, tri-, or poly-substituted with alkyl from C₂ to C₁₂, alkenyl from C₂ to C₁₂, alkynyl from C₂ to C₁₂, or acyl from C₂ to C₁₂. In another aspect, R⁶ can be an O, N, or S mono- or bi-cyclic heterocycle having from two to nine carbon atoms. In yet another aspect, R⁶ can be an O, N, or S mono- or bi-cyclic heterocycle having from two to nine carbon atoms and mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂, where R⁹ is alkyl from C₁ to C₁₂.

In a further more specific aspect of Compound 1, a molecule is provided having the structure as in Compound 20:

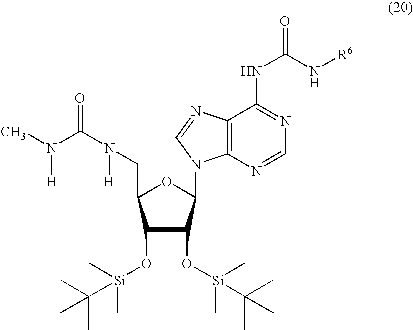

Such a molecule is essentially Compound 1 where R¹ is H, R² is CH₃, R³ is H, R⁴ is H, R⁵ is H, U is H, Z is H, W is O-tert-butyldimethylsilyl, Y is O-tert-butyldimethylsilyl, X¹ is O, X² is O. Additionally, R⁶ is a member selected from a mono-, di-, or tri-cyclic aryl from C₆ to C₁₄, a mono-, di-, or tri-cyclic aryl from C₆ to C₁₄ mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂, where R⁹ is alkyl from C₁ to C₁₂.

In another more specific aspect of Compound 20, a molecule is provided having the structure as in Compound 25, where R⁶ is phenyl:

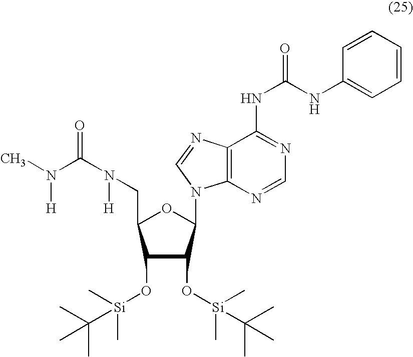

Numerous additional nucleosides having the general structure of Compound 20 are additionally contemplated. For example, in one aspect R⁶ can be a mono-, di-, or tri-cyclic aryl from C₆ to C₁₄. In another aspect, R⁶ can be a mono-, di-, or tri-cyclic aryl from C₆ to C₁₄ mono-, di-, tri-, or poly-substituted with a member selected independently from F, Cl, Br, or I. In yet another aspect, R⁶ can be a mono-, di-, or tri-cyclic aryl from C₆ to C₁₄ mono-, di-, tri-, or poly-substituted with alkoxy (R⁹O—), where R⁹ is alkyl from C₁ to C₁₂. In a further aspect, R⁶ can be a mono-, di-, or tri-cyclic aryl from C₆ to C₁₄ mono-, di-, tri-, or poly-substituted with nitro (NO₂), nitroso (NO), or azido (N₃). In yet a further aspect, R⁶ can be a mono-, di-, or tri-cyclic aryl from C₆ to C₁₄ mono-, di-, tri-, or poly-substituted with alkyl from C₂ to C₁₂, alkenyl from C₂ to C₁₂, alkynyl from C₂ to C₁₂, or acyl from C₂ to C₁₂. In another aspect, R⁶ can be an O, N, or S mono- or bi-cyclic heterocycle having from two to nine carbon atoms. In yet another aspect, R⁶ can be an O, N, or S mono- or bi-cyclic heterocycle having from two to nine carbon atoms and mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂, where R⁹ is alkyl from C₁ to C₁₂.

In yet a further more specific aspect of Compound 1, a molecule is provided having the structure as in Compound 27:

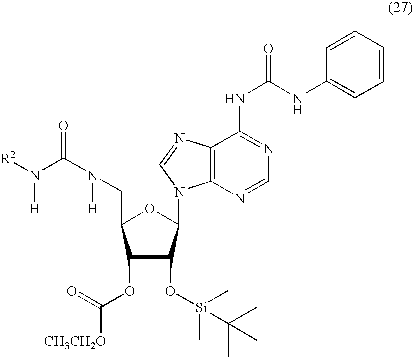

Such a molecule is essentially Compound 1 where R¹ is H, R³ is H, R⁴ is H, R⁵ is H, R⁶ is C₆H₅, U is H, W is CH₃CH₂O₂CCH₂—, Z is H, Y is O-tert-butyldimethylsilyl, X¹ is O, X² is O. Additionally, R² is selected from H, HO—, CH₃O—, CH₃—, HOCH₂CH₂—, HOCH₂CH₂OCH₂CH₂—, NH₂CH₂CH₂—, R⁷NHCH₂CH₂—, (R⁷)₂NCH₂CH₂—, NH₂CH₂CH₂NHCH₂CH₂—, R⁷NHCH₂CH₂NHCH₂CH₂—, (R⁷)₂NCH₂CH₂NHCH₂C₁₋₁₂—, R⁸CO—, or a mono-, di-, or tri-cyclic aryl from C₆ to C₁₄, where R⁷ is an alkyl from C₁ to C₅ and R⁸ is H₂N—, HOHN—, alkyl from C₁ to C₁₀, alkenyl from C₂ to C₁₀, or phenyl.

In another more specific aspect of Compound 1, a molecule is provided having the structure as in Compound 30:

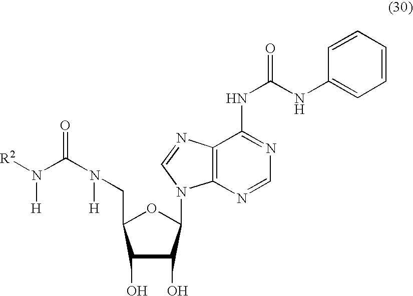

Such a molecule is essentially Compound 1 where R¹ is H, R³ is H, R⁴ is H, R⁵ is H, R⁶ is C₆H₅, U is H, W is OH, Z is H, Y is OH, X¹ is O, and X² is O. Additionally, R² is a member selected from H, HO—, CH₃O—, CH₃—, HOCH₂CH₂—, HOCH₂CH₂OCH₂CH₂—, NH₂CH₂CH₂—, R⁷NHCH₂CH₂—, (R⁷)₂NCH₂CH₂—, NH₂CH₂CH₂NHCH₂CH₂—, R⁷NHCH₂CH₂NHCH₂CH₂—, (R⁷)₂NCH₂CH₂NHCH₂CH₂—, R⁸CO—, or a mono-, di-, or tri-cyclic aryl from C₆ to C₁₄, R⁷ is an alkyl from C₁ to C₅, and R⁸ is H₂N—, HOHN—, alkyl from C₁ to C₁₀, alkenyl from C₂ to C₁₀, or phenyl.

In another more specific aspect of Compound 1, a molecule is provided having the structure as in Compound 29:

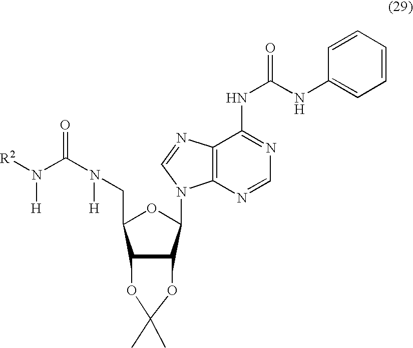

Such a molecule is essentially Compound 1 where R¹ is H, R³ is H, R⁴ is H, R⁵ is H, R⁶ is C₆H₆, U is H, Z is H, W and Y are —OC(CH₃)₂O—, X¹ is O, and X² is O. Additionally, R² is a member selected from H, HO—, CH₃O—, CH₃—, HOCH₂CH₂—, HOCH₂CH₂OCH₂CH₂—, NH₂CH₂CH₂—, R⁷NHCH₂CH₂—, (R⁷)₂NCH₂CH₂—, NH₂CH₂CH₂NHCH₂CH₂—, R⁷NHCH₂CH₂NHCH₂CH₂—, (R⁷)₂NCH₂CH₂NHCH₂CH₂—, R⁸CO—, or a mono-, di-, or tri-cyclic aryl from C₆ to C₁₄, where R⁷ is an alkyl from C₁ to C₅, and R⁸ is H₂N—, HOHN—, alkyl from C₁ to C₁₀, alkenyl from C₂ to C₁₀, or phenyl.

In another aspect of the present invention, a molecule is provided having the structure as in Compound 2:

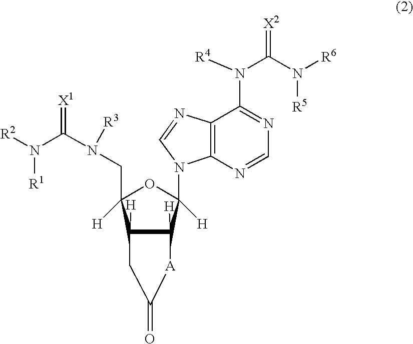

In such molecules, R¹, R², R⁵, and R⁶, are members selected independently from H, HO—, CH₃O—, CH₃—, HOCH₂CH₂—, HOCH₂CH₂OCH₂CH₂—, NH₂CH₂CH₂—, R⁷NHCH₂CH₂—, (R⁷)₂NCH₂CH₂—, NH₂CH₂CH₂NHCH₂CH₂—, R⁷NHCH₂CH₂NHCH₂CH₂—, (R⁷)₂NCH₂CH₂NHCH₂CH₂—, R⁸CO—, a mono-, di-, or tri-cyclic aryl from C₆ to C₁₄, a mono-, di-, or tri-cyclic aryl from C₆ to C₁₄ mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂, where R⁷ is an alkyl from C₁ to C₅, R⁸ is H₂N—, HOHN—, alkyl from C₁ to C₁₀, alkenyl from C₂ to C₁₀, or phenyl, and R⁹ is alkyl from C₁ to C₁₂. Furthermore, R³ and R⁴ include members selected independently from H, HO—, CH₃—, or CH₃CH₂—, and X¹ and X² are members selected independently from O and S. Additionally, A includes a member selected from O, and Ne, where R¹⁰ is H, HO—, CH₃—, or CH₃CH₂—.

In a more specific aspect of Compound 2, a molecule is provided having the structure as in Compound 32:

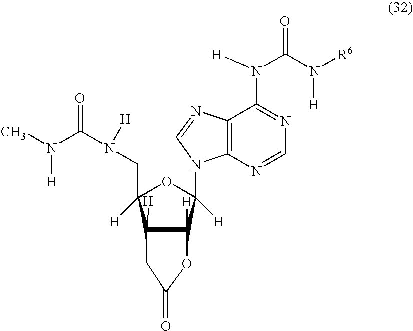

Such a molecule is essentially Compound 2 where R¹ is H, R² is CH₃, R³ is H, R⁴ is H, R⁵ is H, X¹ is O, X² is O, and A is O. Additionally, R⁶ can be a mono-, di-, or tri-cyclic aryl from C₆ to C₁₄.

Numerous additional nucleosides having the general structure of Compound 32 are additionally contemplated. For example, in one aspect R⁶ can be a mono-, di-, or tri-cyclic aryl from C₆ to C₁₄ mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂, and where R⁹ is alkyl from C₁ to C₁₂.

In another more specific aspect of Compound 32, a molecule is provided having the structure as in Compound 33, where R⁶ is phenyl:

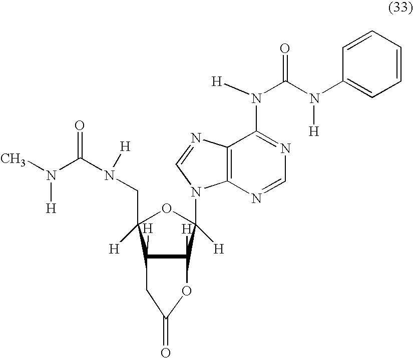

In another more specific aspect of Compound 2, a molecule is provided having the structure as in Compound 39:

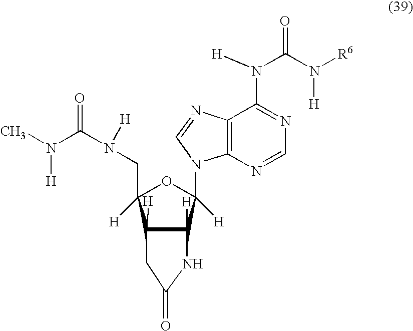

Such a molecule is essentially Compound 2 where R¹ is H, R² is CH₃, R³ is H, R⁴ is H, R⁵ is H, X¹ is O, X² is O, and A is NH. Additionally, R⁶ can be a mono-, di-, or tri-cyclic aryl from C₆ to C₁₄.

Numerous additional nucleosides having the general structure of Compound 39 are additionally contemplated. For example, in one aspect R⁶ is a member selected from a mono-, di-, or tri-cyclic aryl from C₆ to C₁₄ mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂, and where R⁹ is alkyl from C₁ to C₁₂.

In another more specific aspect of Compound 2, a molecule is provided having the structure as in Compound 40:

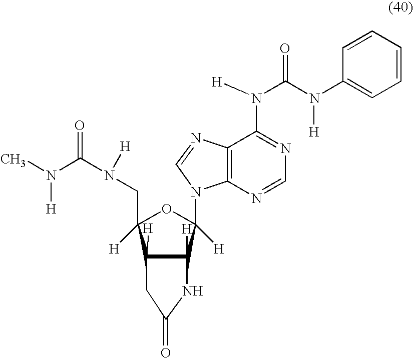

The various nucleosides according to aspects of the present invention may be formulated into compositions useful for the treatment of numerous kinase-related medical conditions. As such, a given nucleoside may be combined with a pharmaceutical carrier for administration to a subject. A variety of excipients may be utilized in the formulation as is well known in the art.

### EXAMPLES

The following examples are provided to promote a more clear understanding of certain embodiments of the present invention, and are in no way meant as a limitation thereon.

### Examples 1-5

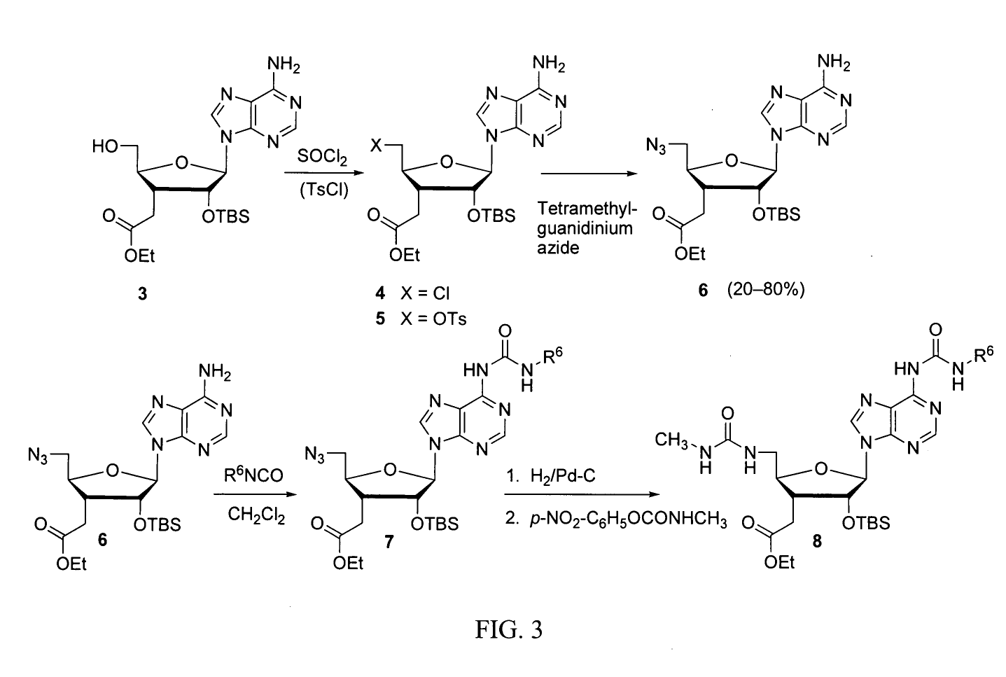

### Synthesis of Compounds 4-8 (FIG. 3)

### Example 1

### Synthesis of 2′-O-(tert-Butyldimethylsilyl)-5′-chloro-3′,5′-dideoxy-3′-[(ethoxycarbonyl)methyl]adenosine (Compound 4)

Thionyl chloride (2 M in CH₂Cl₂, 1.0 mL, 2.0 mmol) is added to a stirred solution of Compound 3 (200 mg, 0.443 mmol; see FIG. 3) and pyridine (100 mg, 1.27 mmol) in CH₂Cl₂ (3.0 mL) at 0° C. The mixture is stirred for 30 min, then allowed to warm to room temperature and stirred overnight. Volatiles are removed under reduced pressure and the residue is partitioned (EtOAc//NaHCO₃(aq)). The organic layer is dried (Na₂SO₄), filtered, and volatiles are removed under reduced pressure. Chromatography (5% MeOH/CH₂Cl₂) gives Compound 4 (62 mg, 30%): UV (MeOH) λ max 260 nm, 2 min 230 nm; ¹H NMR (CDCl₃, 500 MHz) δ 8.35 (s, 1H), 8.18 (s, 1H), 5.97 (s, 1H), 5.59 (br s, 2H), 4.94 (d, J=4.5 Hz, 1H), 4.37-4.34 (m, 1H), 4.12 (q, J=7.4 Hz, 2H), 4.01 (dd, J=3.0, 12.5 Hz, 1H), 3.78 (dd, J=4.3, 12.8 Hz, 1H), 2.85-2.82 (m, 1H), 2.70 (dd, J=9.0, 17.0 Hz, 1H), 2.42 (dd, J=5.8, 16.8 Hz, 1H), 1.26 (t, J=7.3 Hz, 3H), 0.90 (s, 9H), 0.15 (s, 3H), 0.07 (s, 3H); ¹³C NMR (CDCl₃, 50 MHz) δ 171.9, 155.8, 153.2, 138.2, 120.4, 91.3, 82.9, 77.5, 61.1, 45.2, 40.7, 30.1, 25.9, 18.1, 14.3, −4.4, −5.4; MS (FAB) m/z 492.1805 (MNa⁺[C₂₀H₃₂³⁵ClN₅O₄SiNa]=492.1810).

### Example 2

### Synthesis of 2′-O-(tert-Butyldimethylsilyl)-3′-deoxy-3′-[(ethoxycarbonyl)methyl]-5′-O-(p-toluenesulfonyl)adenosine (Compound 5)

Ice-cold CH₂Cl₂ (4.0 mL at 0° C.) is added to a chilled (0° C.) flame-dried flask containing Compound 3 (378 mg, 0.837 mmol; azeotropically dried via evaporation of benzene, 5×20 mL; see FIG. 3), p-toluenesulfonyl-chloride (278 mg, 1.46 mmol), and DMAP (218 mg, 1.78 mmol). The solution is stirred for 24 h at 0° C., then applied directly to a chromatography column and eluted (80% EtOAc/hexanes*EtOAc). Appropriate fractions are pooled and volatiles are removed under reduced pressure (≦20° C.) to give Compound 5 (390 mg, 77%). Compound 5 is not stable at ambient temperature and decomposes upon standing either in solution or as a solid amorphous glass. Characterization is therefore accomplished immediately following isolation, and maximum purities obtained in this way are approximately 90%. Unambiguous characterization by ¹³C NMR is thus complicated by compound instability: ¹H NMR (CDCl₃, 500 MHz) 8, 8.30 (s, 1H), 7.95 (s, 1H), 7.77-7.75 (m, 2H), 7.29-7.28 (m, 2H), 5.91 (d, J=1.0 Hz, 1H), 5.56 (br s, 2H), 4.85 (d, J=4.0 Hz, 1H), 4.37 (dd, J=2.0, 8.5 Hz, 1H), 4.27-4.20 (m, 2H), 4.11 (q, 1=7.2 Hz, 2H), 2.82-2.76 (m, 1H), 2.64 (dd, J=8.8, 16.8 Hz, 1H), 2.42 (s, 3H), 2.32 (dd, J=5.5, 17.0 Hz, 1H), 1.19 (t, J=7.2 Hz, 3H), 0.89 (s, 9H), 0.14 (s, 3H), 0.03 (s, 3H); MS (FAB) m/z 606.2417 (MH⁺[C₂₇H₄₀N₅O₇SSi]=606.2418).

### Example 3

### Synthesis of 5′-Azido-2′-O-(tert-butyldimethylsilyl)-3′,5′-dideoxy-3′-[(ethoxycarbonyl)methyl]adenosine (Compound 6)

Ice-cold CH₂Cl₂ (16 mL at 0° C.) is added to a chilled (0° C.) flame-dried flask containing Compound 3 (360 mg, 0.797 mmol; azeotropically dried via evaporation of benzene, 5×20 mL; see FIG. 3), p-toluenesulfonylchloride (208 mg, 1.10 mmol), and DMAP (208 mg, 1.70 mmol). The solution is stirred for 24 h at 0° C., after which volatiles are removed under reduced pressure (≦20° C.). Tetramethylguanidinium azide (TMGA, 880 mg, 5.56 mmol) and DMF (4 mL) are immediately added and the solution is heated at 65° C. for 7 h. The mixture is cooled to ambient temperature and then vigorously stirred while anhydrous Et₂O (100 mL) is slowly added. Precipitated TMGA is removed by filtering through celite. The white solid mass is triturated, and the filter cake is washed with anhydrous Et₂O to ensure complete transfer of product. Volatiles are removed under reduced pressure (40° C.) and the residue chromatographed (90% EtOAc/hexanes*EtOAc) to give Compound 6 (315 mg, 83%): UV (MeOH) λmax 262 nm, λmin 233 nm; ¹HNMR (CDCl₃, 500 MHz) δ 8.36 (s, 1H), 8.16 (s, 1H), 5.98 (s, 1H), 5.54 (br s, 2H), 4.86 (d, J=5.0 Hz, 1H), 4.22-4.20 (m, 1H), 4.14 (q, J=7.0 Hz, 2H), 3.78 (dd, J=3.3, 13.8 Hz, 1H), 3.61 (dd, J=4.8, 13.8 Hz, 1H), 2.85-2.77 (m, 1H), 2.69 (dd, J=8.3, 16.8 Hz, 1H), 2.37 (dd, J=5.8, 16.8 Hz, 1H), 1.26 (t, J=7.3 Hz, 3H), 0.91 (s, 9H), 0.17 (s, 3H), 0.07 (s, 3H); ¹³C NMR (CDCl₃, 125 MHz) δ 171.6, 155.4, 153.0, 149.4, 138.7, 120.2, 91.1, 82.2, 77.3, 60.9, 52.2, 40.0, 29.9, 25.7, 17.9, 14.1, −4.5, −5.5; MS (FAB) m/z 499.2214 (MNa⁺[C₂₀H₃₂N₈O₄SiNa]=499.2214).

### Example 4

### Synthesis of 5′-Azido-2′-O-(tert-butyldimethylsilyl)-3′,5′-dideoxy-3′-[(ethoxycarbonyl)methyl]-N⁶-(N-R⁶-substitutedcarbamoyl)adenosine (Compound 7)

The general procedure used to prepare Compound 9 (FIG. 4) from Compound 6 can be used to prepare a number of structurally related derivatives typified by the structure of Compound 7. Briefly, R⁶NCO (1.60 mmol) is added to a stirred solution of Compound 6 (1.33 mmol) in CH₂Cl₂ (16 mL). The mixture is stirred at ambient temperature until thin layer chromatography (TLC) indicates complete conversion of Compound 6 to the desired product. The mixture is added directly to a chromatography column and eluted with an appropriate solvent to give Compounds 7.

### Example 5

### Synthesis of 2′-O-(tert-Butyldimethylsilyl)-3′,5′-dideoxy-3′-[(ethoxycarbonyl)methyl]-5′-[(N-methylcarbamoyl)amino]-N⁶-(N-R⁶-substitutedcarbamoyl)adenosine (Compound 8)

The general procedure used to prepare Compound 10 from Compound 9 (both from FIG. 4) can be used to prepare a number of structurally related derivatives typified by the structure of Compound 8. Briefly, a solution of Compound 7 (0.168 mmol) and 10% Pd—C (50 mg) in EtOAc (2 mL) is vigorously stirred for 15 h under an atmosphere of H₂ (balloon pressures). p-Nitrophenyl N-methylcarbamate (45 mg, 0.23 mmol) and anhydrous Na₂CO₃ (45 mg, 0.42 mmol) are added, and the resulting mixture is stirred for 4 h under N₂. Solids are removed via filtration (celite/EtOAc), and volatiles are evaporated under reduced pressure. The crude residue is chromatographed to give Compound 8.

### Examples 6-10

### Synthesis of Compounds 9-13 (FIG. 4)

### Example 6

### Synthesis of 5′-Azido-2′-O-(tert-butyldimethylsilyl)-3′,5′-dideoxy-3′-[(ethoxycarbonyl)methyl]-N⁶-(N-phenylcarbamoyl)adenosine (Compound 9)

Phenylisocyanate (190 mg, 1.60 mmol) is added to a stirred solution of Compound 6 (633 mg, 1.33 mmol) in CH₂Cl₂ (16 mL). The mixture is stirred at ambient temperature until TLC indicates complete conversion of Compound 6 to Compound 9 (5 days). The mixture is added directly to a chromatography column and eluted (10*40% EtOAc/hexanes) to give Compound 9 (755 mg, 95%): UV (MeOH) λmax 279 nm, λmin 243 nm; ¹H NMR (CDCl₃, 500 MHz) δ 11.74 (s, 1H), 8.62 (s, 1H), 8.39 (s, 1H), 8.11 (s, 1H), 7.65 (d, J=8.5 Hz, 2H), 7.39-7.36 (m, 2H), 7.14-7.12 (m, 1H), 6.04 (s, 1H), 4.86 (d, J=5.0 Hz, 1H), 4.24-4.22 (m, 1H), 4.14 (q, J=7.2 Hz, 2H), 3.81 (dd, J=2.8, 13.3 Hz, 1H), 3.63 (dd, J=4.3, 13.3 Hz, 1H), 2.81-2.79 (m, 1H), 2.69 (dd, J=8.5, 17.0 Hz, 1H), 2.39 (dd, J=5.3, 17.3 Hz, 1H), 1.26 (t, J=7.3 Hz, 3H), 0.93 (s, 9H), 0.19 (s, 3H), 0.07 (s, 3H); ¹³C NMR (CDCl₃, 125 MHz) δ 171.5, 151.4, 150.8, 150.0, 149.9, 141.5, 138.1, 129.0, 123.8, 120.2, 91.3, 82.5, 77.5, 60.9, 52.2, 40.1, 29.7, 25.7, 18.0, 14.1, −4.5, −5.5; MS (FAB) m/z 596.2772 (MH⁺[C₂₇H₃₈N₉O₅Si]=596.2765).

### Example 7

### Synthesis of 2′-O-(tert-Butyldimethylsilyl)-3′,5′-dideoxy-3′-[ethoxycarbonyl)methyl]-5′-[(N-methylcarbamoyl)amino]-N⁶-(N-phenylcarbamoyl)adenosine (Compound 10)

A solution of Compound 9 (100 mg, 0.168 mmol) and 10% Pd—C (50 mg) in EtOAc (2 mL) is vigorously stirred for 15 h under an atmosphere of H₂ (balloon pressures). p-Nitrophenyl N-methylcarbamate (45 mg, 0.23 mmol) and anhydrous Na₂CO₃ (45 mg, 0.42 mmol) are added, and the resulting mixture is stirred for 4 h under N₂. Solids are removed via filtration (celite/EtOAc), and volatiles are evaporated under reduced pressure. The crude residue is chromatographed (5*10% MeOH/CH₂Cl₂) to give Compound 10 (101 mg, 96%): UV (MeOH) λmax 279 nm (E 22,700), λmin 242 nm; ¹H NMR (CDCl₃, 500 MHz) δ 12.31 (s, 1H), 10.13 (br s, 1H), 8.86 (s, 1H), 8.64 (s, 1H), 7.57 (d, J=7.5 Hz, 2H), 7.42-7.39 (m, 2H), 7.21-7.18 (m, 1H), 5.94 (s, 1H), 5.78 (t, J=6.3 Hz, 1H), 5.06-5.03 (m, 2H), 4.20 (d, J=10.5 Hz, 1H), 4.11-4.07 (m, 2H), 3.85-3.83 (m, 1H), 3.49 (d, J=13.0 Hz, 1H), 2.79 (dd, J=4.5, 17.0 Hz, 1H), 2.62 (d, J=5.0 Hz, 3H), 2.62-2.50 (m, 1H), 2.49-2.48 (m, 1H), 1.24 (t, J=7.0 Hz, 3H), 0.94 (s, 9H), 0.27 (s, 3H), 0.11 (s, 3H); ¹³C NMR (CDCl₃, 125 MHz) δ 172.0, 159.4, 153.3, 149.9, 149.8, 142.8, 137.3, 129.1, 124.6, 121.2, 92.0, 84.7, 77.2, 60.3, 39.7, 38.5, 28.8, 26.7, 25.7, 17.9, 14.0, −4.3, −5.8; MS (FAB) m/z 649.2899 (MNa⁺[C₂₉H₄₂N₈O₆SiNa]=649.2894).

### Example 8

### Synthesis of 5-Azido-2′-O-(tert-butyldimethylsilyl)-3′-(carboxymethyl)-3′,5′-dideoxyadenosine (Compound 11)

NaOH (200 μL, 5.0 M, 1.0 mmol) and MeOH (400 μL) are added to a stirred solution of Compound 6 (150 mg, 0.315 mmol) in THF (2 mL). The mixture is stirred at ambient temperature until starting material has been converted to baseline product (6 h, TLC). Volatiles are removed under reduced pressure (≦20° C.) and the crude material is partitioned (CH₂Cl₂//H₂O). Ice is added and the pH is carefully adjusted to ≈3 via dropwise addition of 1% HCl(aq). The aqueous layer is washed (CH₂Cl₂, 5×) until the organic layer is UV transparent (TLC). The combined organic layers are dried (Na₂SO₄), filtered, and evaporated under reduced pressure 20° C.) to give Compound 11 (120 mg, 85%): UV (MeOH) λmax 260 nm, λmin 233 nm; ¹H NMR (CDCl₃, 500 MHz) δ 8.32 (s, 1H), 8.25 (s, 1H), 7.27 (br s, 2H), 6.02 (s, 1H), 4.76 (d, J=4.0 Hz, 1H), 4.25 (dd, J=6.5, 10.5 Hz, 1H), 3.86 (d, J=13.0 Hz, 1H), 3.63 (dd, J=3.5, 13.5 Hz, 1H), 2.83-2.80 (m, 1H), 2.71 (dd, J=8.5, 17.0 Hz, 1H), 2.42 (dd, J=4.8, 17.3 Hz, 1H), 0.93 (s, 9H), 0.21 (s, 3H), 0.10 (s, 3H); ¹³C NMR (CDCl₃, 125 MHz) δ 176.1, 155.4, 151.8, 148.9, 138.8, 118.9, 91.1, 82.5, 77.9, 51.9, 39.8, 30.2, 29.7, 25.7, 18.0, −4.5, −5.5; MS (FAB) m/z 471.1902 (MNa⁺[C₁₈H₂₈N₈O₄SiNa]=471.1901).

### Example 9

### Synthesis of 5′-Azido-2′-O-(tert-butyldimethylsilyl)-3′,5′-dideoxy-3′-[(N-methoxy-N-methyl carboxamido)methyl]adenosine (Compound 12)

Carbonyl diimidazole (500 μL of 0.36 M solution in CH₂Cl₂, 29 mg, 0.18 mol) is added to a stirred solution of Compound 11 (50 mg, 0.112 mmol) in CH₂Cl₂ (1.0 mL) at 0° C. The ice-bath is removed and the reaction is allowed to warm to ambient temperature for 1 h. N,O-Dimethylhydroxylamine hydrochloride (18 mg, 0.19 mmol) and Et₃N (82 mg, 0.82 mmol) are added and the reaction is followed by TLC (24 h). Chromatography (5% MeOH/EtOAc) gave Compound 12 (46 mg, 84%): UV (MeOH) λmax 260 nm, λmin 230 nm; NMR (CDCl₃, 500 MHz) δ 8.35 (s, 1H), 8.16 (s, 1H), 5.99 (d, J=2.0 Hz, 1H), 5.67 (br s, 2H), 4.87-4.86 (m, 1H), 4.25-4.22 (m, 1H), 3.77 (dd, J=2.8, 13.3 Hz, 1H), 3.70 (s, 3H), 3.65 (dd, J=4.5, 13.5 Hz, 1H), 3.16 (s, 3H), 2.85-2.83 (m, 2H), 2.60-2.52 (m, 1H), 0.90 (s, 9H), 0.11 (s, 3H), 0.02 (s, 3H); ¹³C NMR (CDCl₃, 125 MHz) δ 172.6, 155.7, 153.2, 149.8, 138.8, 120.3, 91.0, 82.9, 77.8, 61.5, 53.0, 39.9, 32.5, 28.4, 26.0, 18.2, −4.40, −5.10; MS (FAB) m/z 514.2327 (MNa⁺[C₂₀H₃₃N₉O₄SiNa]=514.2323).

### Example 10

### Synthesis of 2′-O-(tert-Butyldimethylsilyl)-3′,5′-dideoxy-3′-[(N-methoxy-N-methylcarboxamido) methyl]-5′-[(N-methylcarbamoyl)amino]-N⁶-(N-phenylcarbamoyl)adenosine (Compound 13)

A solution of Compound 12 (50 mg, 0.082 mmol) and 10% Pd—C (50 mg) in EtOAc (1 mL) is vigorously stirred for 18 h under an atmosphere of H₂ (balloon pressures). p-Nitrophenyl N-methyl-carbamate (25 mg, 0.13 mmol) and anhydrous Na₂CO₃ (50 mg, 0.47 mmol) are added, and the resulting mixture is stirred for 4 h under N₂. Solids are removed via filtration (celite/EtOAc), volatiles are evaporated under reduced pressure, and the residue is chromatographed (10% MeOH/EtOAc) to give Compound 13 (33 mg, 63%): UV (MeOH) λmax 279 nm (ε 22,200), λmin 245 nm; ¹H NMR (CDCl₃, 500 MHz) δ 12.32 (s, 1H), 10.14 (br s, 1H), 8.90 (s, 1H), 8.61 (s, 1H), 7.58 (d, J=7.5 Hz, 2H), 7.40 (t, J=7.5 Hz, 2H), 7.19-7.16 (m, 1H), 5.96 (s, 1H), 5.85 (br s, 1H), 5.07 (d, J=4.0 Hz, 1H), 5.02 (d, J=3.5 Hz, 1H), 4.25 (d, J=10.5 Hz, 1H), 3.78-3.75 (m, 1H), 3.73 (s, 3H), 3.58 (d, J=11.5 Hz, 1H), 3.13 (s, 3H), 2.78 (d, J=5.0 Hz, 2H), 2.61 (d, J=4.5 Hz, 3H), 2.50-2.46 (m, 1H), 0.94 (s, 9H), 0.28 (s, 3H), 0.10 (s, 3H); ¹³C NMR (CDCl₃, 125 MHz) δ 172.7, 159.3, 153.2, 150.04, 150.01, 149.9, 142.8, 137.5, 129.1, 124.5, 121.2, 92.1, 84.8, 77.6, 61.1, 40.3, 38.4, 32.1, 29.7, 26.8, 25.8, 18.0, −4.4, −5.5; MS (ES) m/z 642.3182 (MH⁺[C₂₉H₄₄N₉O₆Si]=642.3184).

### Examples 11-17

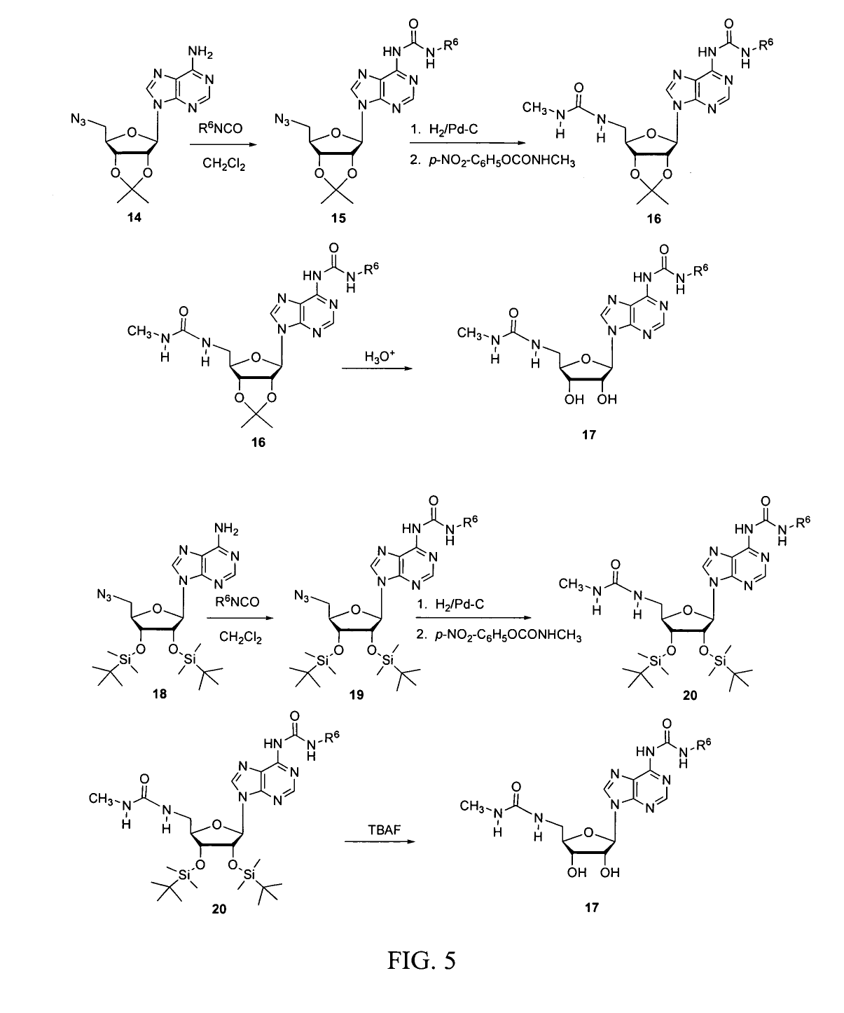

### Synthesis of Compounds 14-20 (FIG. 5)

### Example 11

### Synthesis of 5′-azido-5′-deoxy-2′,3′-bis-O-isopropylideneadenosine (Compound 14)

A solution of 5′-azido-5′-deoxyadenosine (1.0 g, 3.42 mmol) and HClO₄ (1.0 mL, conc.) in dry acetone (1.0 L) is stirred vigorously at room temperature until TLC indicates that all of the starting material has been converted to Compound 14. Solid K₂CO₃ (anhydrous) is added to neutralize the acid. Solids are removed via filtration and volatiles are removed under reduced pressure to give Compound 14.

### Example 12

### Synthesis of 5′-azido-5′-deoxy-2′,3′-bis-O-isopropylidene-N⁶-(N-R⁶-substitutedcarbamoyl)adenosine (Compound 15)

R⁶NCO (1.2 equiv.) is added to a stirred solution of Compound 14 in CH₂Cl₂. The mixture is stirred at ambient temperature until TLC indicates complete conversion of Compound 14 to desired product. The mixture is added directly to a chromatography column and eluted with an appropriate solvent to give Compound 15.

### Example 13

### Synthesis of 5′-Deoxy-2′,3′-bis-O-isopropylidene-5′-[(N-methylcarbamoyl)amino]-N⁶-(N-R⁶-substitutedcarbamoyl)adenosine (Compound 16)

A solution of Compound 15 (0.168 mmol) and 10% Pd—C (50 mg) in EtOAc (2 mL) is vigorously stirred for 15 h under an atmosphere of H₂ (balloon pressures). p-Nitrophenyl N-methylcarbamate (45 mg, 0.23 mmol) and anhydrous Na₂CO₃ (45 mg, 0.42 mmol) are added, and the resulting mixture is stirred for 4 h under N₂. Solids are removed via filtration (celite/EtOAc), and volatiles are evaporated under reduced pressure. The crude residue is chromatographed to give Compound 16.

### Example 14

### Synthesis of 5′-1 (N-methylcarbamoyl)amino-1-N⁶-(N-R⁶-substitutedcarbamoyl)adenosine (17)

Method A: A solution of Compound 16 and aqueous acid is vigorously stirred until TLC indicates complete conversion of Compound 16 to Compound 17. Solvents are evaporated and the crude residue is chromatographed to give Compound 17.

Method B: A solution of Compound 20 and tetrabutylammonium fluoride (TBAF, 2.2 equiv.) in THF is stirred until TLC indicates complete conversion of Compound 20 to Compound 17. Solvents are evaporated and the crude residue is chromatographed to give Compound 17.

### Example 15

### Synthesis of 5′-azido-5′-deoxy-2′,3′-bis-O-(tert-butyldimethylsilyl)adenosine (Compound 18)

A solution of 5′-azido-5′-deoxyadenosine is treated with tert-butyldimethylsilylchloride (2.5 equiv.) and imidazole (5.0 equiv.) in dried pyridine. The mixture is stirred protected from moisture until TLC indicates complete conversion of starting material to Compound 18. Volatiles are removed under reduced pressure and the crude residue is purified by chromatography to give Compound 18.

### Example 16

### Synthesis of 5′-azido-2′,3′-bis-O-(tert-butyldimethylsilyl)-5′-deoxy-N⁶-(N-R⁶-substitutedcarbamoyl)-adenosine (Compound 19)

R⁶NCO (1.2 equiv.) is added to a stirred solution of Compound 18 in CH₂Cl₂. The mixture is stirred at ambient temperature until TLC indicates complete conversion of Compound 18 to desired product. The mixture is added directly to a chromatography column and eluted with an appropriate solvent to give Compounds 19.

### Example 17

### Synthesis of 2′,3′-Bis-O-(tert-butyldimethylsilyl)-5′-deoxy-5′-[(N-methylcarbamoyl)amino]-N⁶-(N-R⁶-substitutedcarbamoyl)adenosine (Compound 20)

A solution of Compound 19 (0.168 mmol) and 10% Pd—C (50 mg) in EtOAc (2 mL) is vigorously stirred for 15 h under an atmosphere of H₂ (balloon pressures). p-Nitrophenyl N-methylcarbamate (45 mg, 0.23 mmol) and anhydrous Na₂CO₃ (45 mg, 0.42 mmol) are added, and the resulting mixture is stirred for 4 h under N₂. Solids are removed via filtration (celite/EtOAc), and volatiles are evaporated under reduced pressure. The crude residue is chromatographed to give Compounds 20.

### Examples 18-22

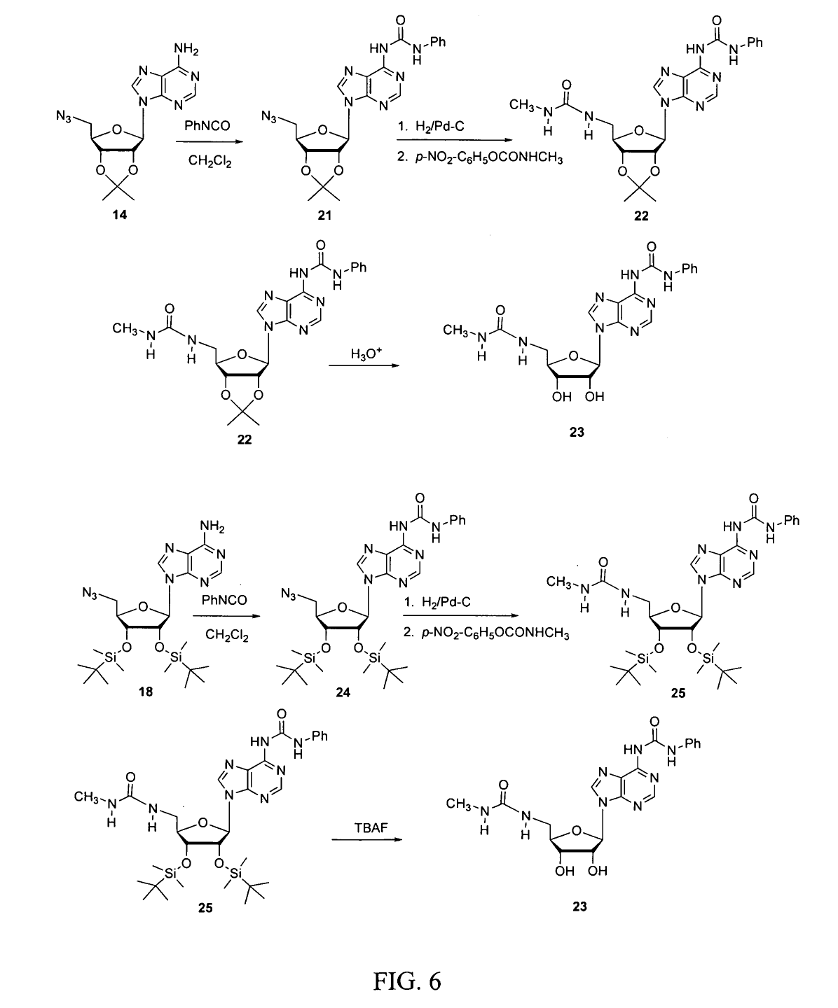

### Synthesis of Compounds 21-25 (FIG. 6)

### Example 18

### Synthesis of 5′-azido-5′-deoxy-2′,3′-bis-O-isopropylidene-N⁶-(N-phenylsubstitutedcarbamoyl)adenosine (Compound 21)

PhNCO (1.2 equiv.) is added to a stirred solution of Compound 14 in CH₂Cl₂. The mixture is stirred at ambient temperature until TLC indicates complete conversion of Compound 14 to Compound 21. The mixture is added directly to a chromatography column and eluted with an appropriate solvent to give Compound 21.

### Example 19

### Synthesis of 5′-Deoxy-2′,3′-bis-O-isopropylidene-5′-[(N-methylcarbamoyl)amino]-N⁶-(N-phenylcarbamoyl)adenosine (Compound 22)

A solution of Compound 21 (0.168 mmol) and 10% Pd—C (50 mg) in EtOAc (2 mL) is vigorously stirred for 15 h under an atmosphere of H₂ (balloon pressures). p-Nitrophenyl N-methylcarbamate (45 mg, 0.23 mmol) and anhydrous Na₂CO₃ (45 mg, 0.42 mmol) are added, and the resulting mixture is stirred for 4 h under N₂. Solids are removed via filtration (celite/EtOAc), and volatiles are evaporated under reduced pressure. The crude residue is chromatographed to give Compound 22.

### Example 20

### Synthesis of 5′-[(N-methylcarbamoyl)amino]-N⁶-(N-phenylcarbamoyl)adenosine (Compound 23)

Method A: A solution of Compound 22 and aqueous acid is vigorously stirred in an appropriate solvent until TLC indicates complete conversion of Compound 22 to Compound 23. Solvents are evaporated and the crude residue is chromatographed to give Compound 23.

Method B: A solution of Compound 25 and tetrabutylammonium fluoride (TBAF, 2.2 equiv.) in THF is stirred until TLC indicates complete conversion of Compound 25 to Compound 23. Solvents are evaporated and the crude residue is chromatographed to give Compound 23.

### Example 21

### Synthesis of 5′-azido-2′,3′-bis-O-(tert-butyldimethylsilyl)-5′-deoxy-N⁶-(N-phenylcarbamoyl)adenosine (Compound 24)

PhNCO (1.2 equiv.) is added to a stirred solution of Compound 18 in CH₂Cl₂. The mixture is stirred at ambient temperature until TLC indicates complete conversion of Compound 18 to Compound 24. The mixture is added directly to a chromatography column and eluted with an appropriate solvent to give Compound 24.

### Example 22

### Synthesis of 2′,3′-Bis-O-(tert-butyldimethylsilyl)-5-deoxy-5′-[(N-methylcarbamoyl)amino]-N⁶-(N-phenylcarbamoyl)adenosine (Compound 25)

A solution of Compound 24 (0.168 mmol) and 10% Pd—C (50 mg) in EtOAc (2 mL) is vigorously stirred for 15 h under an atmosphere of H₂ (balloon pressures). p-Nitrophenyl N-methylcarbamate (45 mg, 0.23 mmol) and anhydrous Na₂CO₃ (45 mg, 0.42 mmol) are added, and the resulting mixture is stirred for 4 h under N₂. Solids are removed via filtration (celite/EtOAc), and volatiles are evaporated under reduced pressure. The crude residue is chromatographed to give Compound 25.

### Example 23

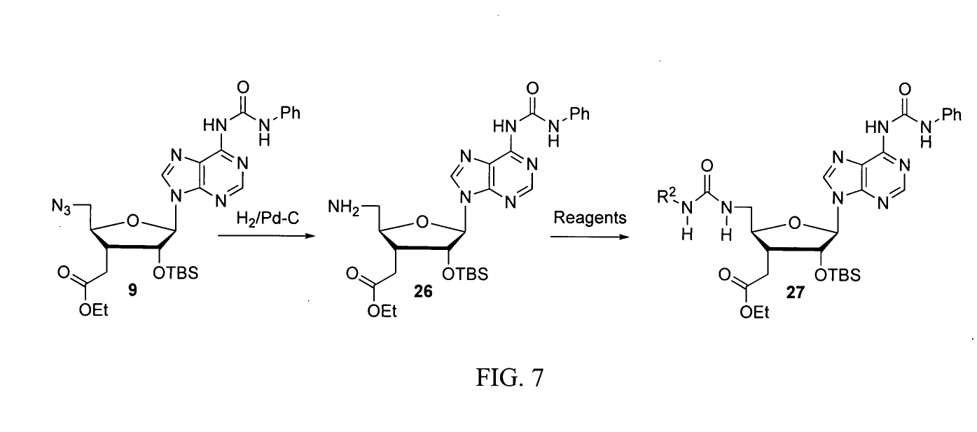

### Synthesis of Compounds 27 (FIG. 7)

Table 1 shows Compounds 27 that can be synthesized according to the methods described herein. Table 2a-d show lists of chemical reactions from Compounds 26 to Compounds 27 listed in Table 1.

TABLE 1
Compounds 27

| Compound | R² |
|---|---|
| 27-1 | NH₂ |
| 27-2 | NHOH |
| 27-3 | NHOCH₃ |
| 27-4 | NHCH₂CH₂OH |
| 27-5 | NHCH₂CH₂OCH₂CH₂OH |
| 27-6 | NHCH₂CH₂NH₂ |
| 27-7 | NHCH₂CH₂NH(CH₃) |
| 27-8 | NHCH₂CH₂NH(CH₂CH₃) |
| 27-9 | NHCH₂CH₂NH(CH₂CH₂CH₃) |
| 27-10 | NHCH₂CH₂NH(CH₂CH₂CH₂CH₃) |
| 27-11 | NHCH₂CH₂NH(CH₂CH₂CH₂CH₂CH₃) |
| 27-12 | NHCH₂CH₂N(CH₃)₂ |
| 27-13 | NHCH₂CH₂NCH₃(CH₂CH₃) |
| 27-14 | NHCH₂CH₂NCH₃(CH₂CH₂CH₃) |
| 27-15 | NHCH₂CH₂NCH₃(CH₂CH₂CH₂CH₃) |
| 27-16 | NHCH₂CH₂N(CH₂CH₃)(CH₂CH₂CH₃) |
| 27-17 | NHCH₂CH₂N(CH₂CH₃)(CH₂CH₃) |
| 27-18 | NHCH₂CH₂N(CH₂CH₂CH₂CH₂) |
| 27-19 | NHCH₂CH₂NHCH₂CH₂NH₂ |
| 27-20 | NHCH₂CH₂NHCH₂CH₂NHCH₃ |
| 27-21 | NHCH₂CH₂NHCH₂CH₂NHCH₂CH₃ |
| 27-22 | NHCH₂CH₂NHCH₂CH₂NHCH₂CH₂CH₃ |
| 27-23 | NHCH₂CH₂NHCH₂CH₂NHCH₂CH₂CH₂CH₃ |
| 27-24 | NHCH₂CH₂NHCH₂CH₂NHCH₂CH₂CH₂CH₂CH₃ |
| 27-25 | NHCH₂CH₂NHCH₂CH₂N(CH₃)₂ |
| 27-26 | NHCH₂CH₂NHCH₂CH₂NCH₃(CH₂CH₃) |
| 27-27 | NHCH₂CH₂NHCH₂CH₂NCH₃(CH₂CH₂CH₃) |
| 27-28 | NHCH₂CH₂NHCH₂CH₂NCH₃(CH₂CH₂CH₂CH₃) |
| 27-29 | NHCH₂CH₂NHCH₂CH₂N(CH₂CH₂CH₃)(CH₂CH₃) |
| 27-30 | NHCH₂CH₂NHCH₂CH₂N(CH₂CH₃)₂ |
| 27-31 | NHCH₂CH₂NHCH₂CH₂N(CH₂CH₂CH₂CH₂) |
| 27-32 | NHCH₂CH₂NHCH₂CH₂NHCH₂CH₂NH₂ |
| 27-33 | CONH₂ |
| 27-34 | CONHOH |
| 27-35 | COCH₃ |
| 27-36 | COCH₂CH₃ |
| 27-37 | COCH₂CH₂CH₃ |
| 27-38 | COCH₂CH₂CH₂CH₃ |
| 27-39 | COCH₂CH₂CH₂CH₂CH₃ |
| 27-40 | COCH₂CH₂CH₂CH₂CH₂CH₃ |
| 27-41 | COCH₂CH₂CH₂CH₂CH₂CH₂CH₃ |
| 27-42 | COCH₂CH₂CH₂CH₂CH₂CH₂CH₂CH₃ |
| 27-43 | COCH₂CH₂CH₂CH₂CH₂CH₂CH₂CH₂CH₃ |
| 27-44 | COCH₂CH₂CH₂CH₂CH₂CH₂CH₂CH₂CH₂CH₃ |
| 27-45 | COCH═CH₂ |
| 27-46 | COCH═CHCH₃ |
| 27-47 | COCH═CHCH₂CH₃ |
| 27-48 | COCH═CHCH₂CH₂CH₃ |
| 27-49 | COCH═CHCH₂CH₂CH₂CH₃ |
| 27-50 | COCH═CHCH₂CH₂CH₂CH₂CH₃ |
| 27-51 | COCH═CHCH₂CH₂CH₂CH₂CH₂CH₃ |
| 27-52 | COCH═CHCH₂CH₂CH₂CH₂CH₂CH₂CH₃ |
| 27-53 | COCH═CHCH₂CH₂CH₂CH₂CH₂CH₂CH₂CH₃ |
| 27-54 | COC₆H₅ |
| 27-55 | C₆H₅ |
| 27-56 | C₁₀H₇ (napthalen-1-yl) |
| 27-57 | C₁₀H₇ (napthalen-2-yl) |
| 27-58 | C₁₄H₉ (anthracen-1-yl) |
| 27-59 | C₁₄H₉ (anthracen-2-yl) |
| 27-60 | C₁₄H₉ (anthracen-9-yl) |

TABLE 2a
Compounds 27 Reactions

| | | |
|---|---|---|
| 26 | 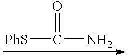 | 27-1 |
| 26 | 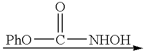 | 27-2 |
| 26 | 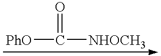 | 27-3 |
| 26 | 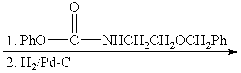 | 27-4 |
| 26 | 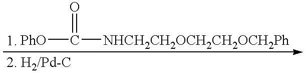 | 27-5 |
| 26 | 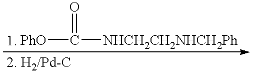 | 27-6 |
| 26 | 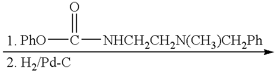 | 27-7 |
| 26 | 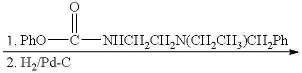 | 27-8 |
| 26 | 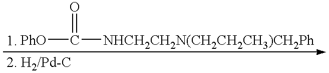 | 27-9 |
| 26 | 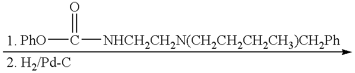 | 27-10 |
| 26 | 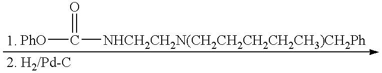 | 27-11 |
| 26 | 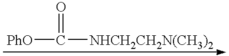 | 27-12 |
| 26 | 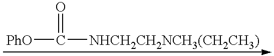 | 27-13 |
| 26 | 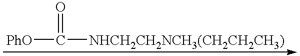 | 27-14 |
| 26 | 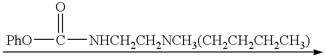 | 27-15 |

TABLE 2b
Compounds 27 Reactions

| | | |
|---|---|---|
| 26 | 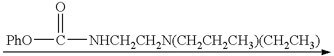 | 27-16 |
| 26 | 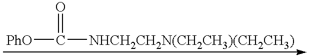 | 27-17 |
| 26 | 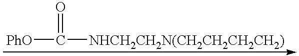 | 27-18 |
| 26 | 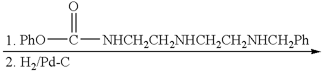 | 27-19 |
| 26 | 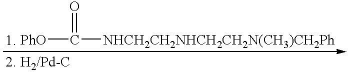 | 27-20 |
| 26 | 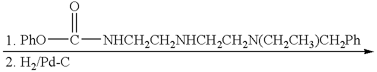 | 27-21 |
| 26 | 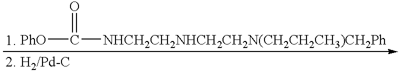 | 27-22 |
| 26 | 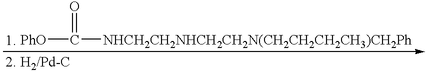 | 27-23 |
| 26 | 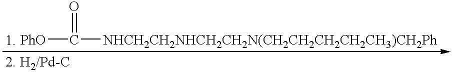 | 27-24 |
| 26 | 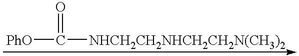 | 27-25 |
| 26 | 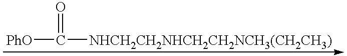 | 27-26 |
| 26 | 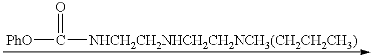 | 27-27 |
| 26 | 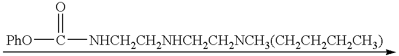 | 27-28 |
| 26 | 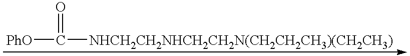 | 27-29 |
| 26 | 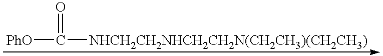 | 27-30 |

TABLE 2c
Compounds 27 Reactions

| | | |
|---|---|---|
| 26 | 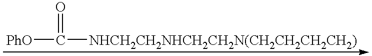 | 27-31 |
| 26 | 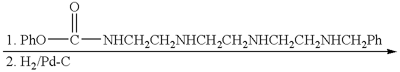 | 27-32 |
| 26 | 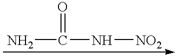 | 27-33 |
| 26 | 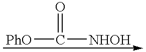 | 27-34 |
| 26 | 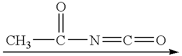 | 27-35 |
| 26 | 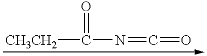 | 27-36 |
| 26 | 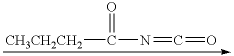 | 27-37 |
| 26 | 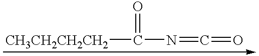 | 27-38 |
| 26 | 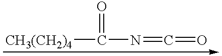 | 27-39 |
| 26 | 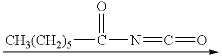 | 27-40 |
| 26 | 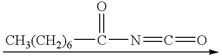 | 27-41 |
| 26 | 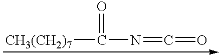 | 27-42 |
| 26 | 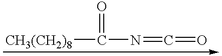 | 27-43 |
| 26 | 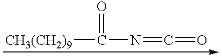 | 27-44 |
| 33 | 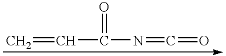 | 27-45 |

TABLE 2d
Compounds 27 Reactions

| | | |
|---|---|---|
| 26 | 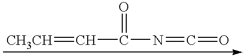 | 27-46 |
| 26 | 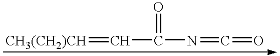 | 27-47 |
| 26 | 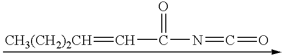 | 27-48 |
| 26 | 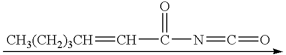 | 27-49 |
| 26 | 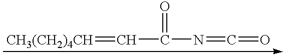 | 27-50 |
| 26 | 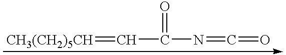 | 27-51 |
| 26 | 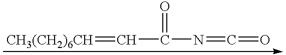 | 27-52 |
| 26 | 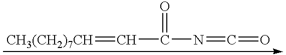 | 27-53 |
| 26 | 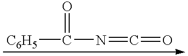 | 27-54 |
| 26 | 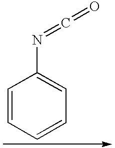 | 27-55 |
| 26 | 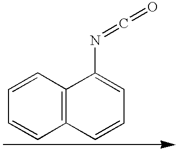 | 27-56 |
| 26 | 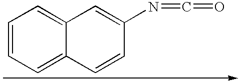 | 27-57 |
| 26 | 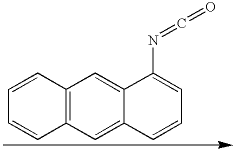 | 27-58 |
| 26 | 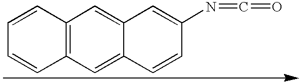 | 27-59 |
| 26 | 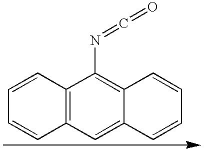 | 27-60 |

### Example 24

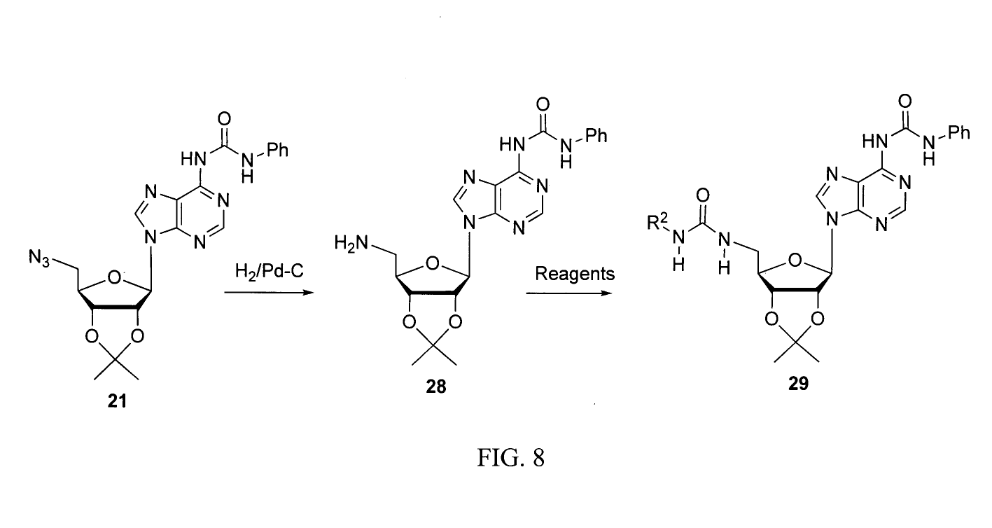

### Synthesis of Compounds 29 (FIG. 8)

Table 3 shows Compounds 29 that can be synthesized according the methods described herein. Table 4a-d show lists of chemical reactions from Compounds 28 to Compounds 29 listed in Table 3.

TABLE 3
Compounds 29

| Compound | R² |
|---|---|
| 29-1 | NH₂ |
| 29-2 | NHOH |
| 29-3 | NHOCH₃ |
| 29-4 | NHCH₂CH₂OH |
| 29-5 | NHCH₂CH₂OCH₂CH₂OH |
| 29-6 | NHCH₂CH₂NH₂ |
| 29-7 | NHCH₂CH₂NH(CH₃) |
| 29-8 | NHCH₂CH₂NH(CH₂CH₃) |
| 29-9 | NHCH₂CH₂NH(CH₂CH₂CH₃) |
| 29-10 | NHCH₂CH₂NH(CH₂CH₂CH₂CH₃) |
| 29-11 | NHCH₂CH₂NH(CH₂CH₂CH₂CH₂CH₃) |
| 29-12 | NHCH₂CH₂N(CH₃)₂ |
| 29-13 | NHCH₂CH₂NCH₃(CH₂CH₃) |
| 29-14 | NHCH₂CH₂NCH₃(CH₂CH₂CH₃) |
| 29-15 | NHCH₂CH₂NCH₃(CH₂CH₂CH₂CH₃) |
| 29-16 | NHCH₂CH₂N(CH₂CH₃)(CH₂CH₂CH₃) |
| 29-17 | NHCH₂CH₂N(CH₂CH₃)(CH₂CH₃) |
| 29-18 | NHCH₂CH₂N(CH₂CH₂CH₂CH₂) |
| 29-19 | NHCH₂CH₂NHCH₂CH₂NH₂ |
| 29-20 | NHCH₂CH₂NHCH₂CH₂NHCH₃ |
| 29-21 | NHCH₂CH₂NHCH₂CH₂NHCH₂CH₃ |
| 29-22 | NHCH₂CH₂NHCH₂CH₂NHCH₂CH₂CH₃ |
| 29-23 | NHCH₂CH₂NHCH₂CH₂NHCH₂CH₂CH₂CH₃ |
| 29-24 | NHCH₂CH₂NHCH₂CH₂NHCH₂CH₂CH₂CH₂CH₃ |
| 29-25 | NHCH₂CH₂NHCH₂CH₂N(CH₃)₂ |
| 29-26 | NHCH₂CH₂NHCH₂CH₂NCH₃(CH₂CH₃) |
| 29-27 | NHCH₂CH₂NHCH₂CH₂NCH₃(CH₂CH₂CH₃) |
| 29-28 | NHCH₂CH₂NHCH₂CH₂NCH₃(CH₂CH₂CH₂CH₃) |
| 29-29 | NHCH₂CH₂NHCH₂CH₂N(CH₂CH₂CH₃)(CH₂CH₃) |
| 29-30 | NHCH₂CH₂NHCH₂CH₂N(CH₂CH₃)₂ |
| 29-31 | NHCH₂CH₂NHCH₂CH₂N(CH₂CH₂CH₂CH₂) |
| 29-32 | NHCH₂CH₂NHCH₂CH₂NHCH₂CH₂NH₂ |
| 29-33 | CONH₂ |
| 29-34 | CONHOH |
| 29-35 | COCH₃ |
| 29-36 | COCH₂CH₃ |
| 29-37 | COCH₂CH₂CH₃ |
| 29-38 | COCH₂CH₂CH₂CH₃ |
| 29-39 | COCH₂CH₂CH₂CH₂CH₃ |
| 29-40 | COCH₂CH₂CH₂CH₂CH₂CH₂ |
| 29-41 | COCH₂CH₂CH₂CH₂CH₂CH₂CH₃ |
| 29-42 | COCH₂CH₂CH₂CH₂CH₂CH₂CH₂CH₃ |
| 29-43 | COCH₂CH₂CH₂CH₂CH₂CH₂CH₂CH₂CH₃ |
| 29-44 | COCH₂CH₂CH₂CH₂CH₂CH₂CH₂CH₂CH₂CH₃ |
| 29-45 | COCH=CH₂ |
| 29-46 | COCH=CHCH₃ |
| 29-47 | COCH=CHCH₂CH₃ |
| 29-48 | COCH=CHCH₂CH₂CH₃ |
| 29-49 | COCH=CHCH₂CH₂CH₂CH₃ |
| 29-50 | COCH=CHCH₂CH₂CH₂CH₂CH₃ |
| 29-51 | COCH=CHCH₂CH₂CH₂CH₂CH₂CH₃ |
| 29-52 | COCH=CHCH₂CH₂CH₂CH₂CH₂CH₂CH₃ |
| 29-53 | COCH=CHCH₂CH₂CH₂CH₂CH₂CH₂CH₂CH₃ |
| 29-54 | COC₆H₅ |
| 29-55 | C₆H₅ |
| 29-56 | C₁₀H₇ (napthalen-1-yl) |
| 29-57 | C₁₀H₇ (napthalen-2-yl) |
| 29-58 | C₁₄H₉ (anthracen-1-yl) |
| 29-59 | C₁₄H₉ (anthracen-2-yl) |
| 29-60 | C₁₄H₉ (anthracen-9-yl) |

TABLE 4a
Compounds 29 Reactions

| | | |
|---|---|---|
| 28 | 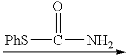 | 29-1 |
| 28 | 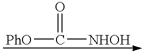 | 29-2 |
| 28 | 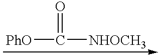 | 29-3 |
| 28 | 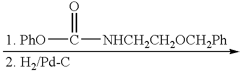 | 29-4 |
| 28 | 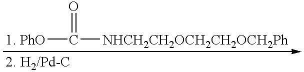 | 29-5 |
| 28 | 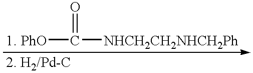 | 29-6 |
| 28 | 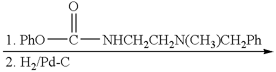 | 29-7 |
| 28 | 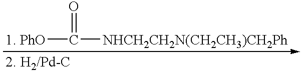 | 29-8 |
| 28 | 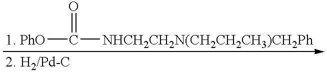 | 29-9 |
| 28 | 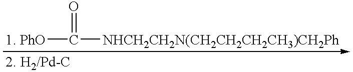 | 29-10 |
| 28 | 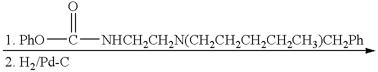 | 29-11 |
| 28 | 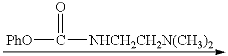 | 29-12 |
| 28 | 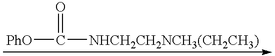 | 29-13 |
| 28 | 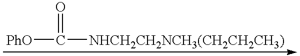 | 29-14 |
| 28 | 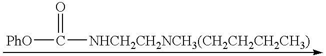 | 29-15 |

TABLE 4b
Compounds 29 Reactions

| | | |
|---|---|---|
| 28 | 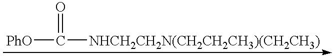 | 29-16 |
| 28 | 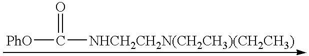 | 29-17 |
| 28 | 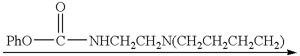 | 29-18 |
| 28 | 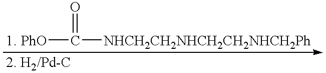 | 29-19 |
| 28 | 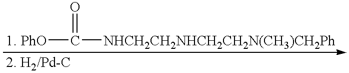 | 29-20 |
| 28 | 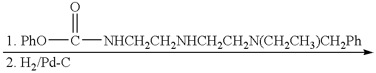 | 29-21 |
| 28 | 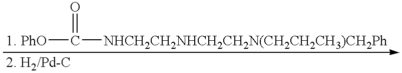 | 29-22 |
| 28 | 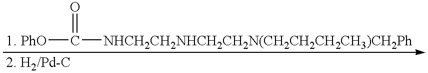 | 29-23 |
| 28 | 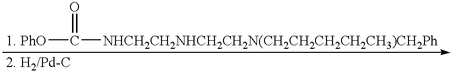 | 29-24 |
| 28 | 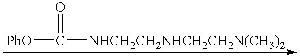 | 29-25 |
| 28 | 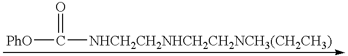 | 29-26 |
| 28 | 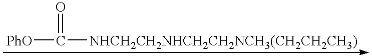 | 29-27 |
| 28 | 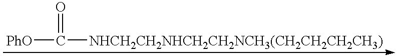 | 29-28 |
| 28 | 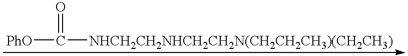 | 29-29 |
| 28 | 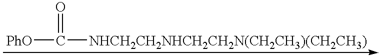 | 29-30 |

TABLE 4c
Compounds 29 Reactions

| | | |
|---|---|---|
| 28 | 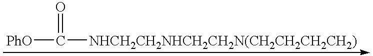 | 29-31 |
| 28 | 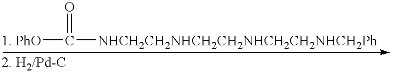 | 29-32 |
| 28 | 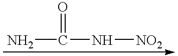 | 29-33 |
| 28 | 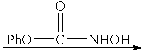 | 29-34 |
| 28 | 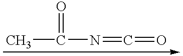 | 29-35 |
| 28 | 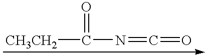 | 29-36 |
| 28 | 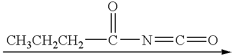 | 29-37 |
| 28 | 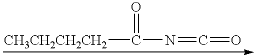 | 29-38 |
| 28 | 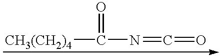 | 29-39 |
| 28 | 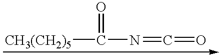 | 29-40 |
| 28 | 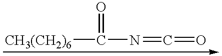 | 29-41 |
| 28 | 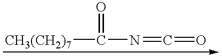 | 29-42 |
| 28 | 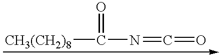 | 29-43 |
| 28 | 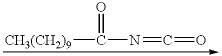 | 29-44 |
| 28 | 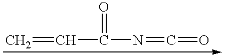 | 29-45 |

TABLE 4d
Compounds 29 Reactions

| | | |
|---|---|---|
| 28 | 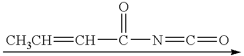 | 29-46 |
| 28 | 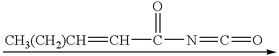 | 29-47 |
| 28 | 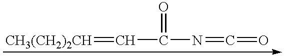 | 29-48 |
| 28 | 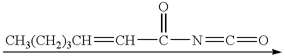 | 29-49 |
| 28 | 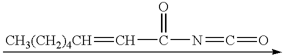 | 29-50 |
| 28 | 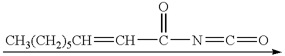 | 29-51 |
| 28 | 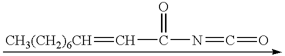 | 29-52 |
| 28 | 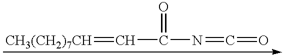 | 29-53 |
| 28 | 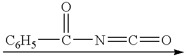 | 29-54 |
| 28 | 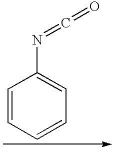 | 29-55 |
| 28 | 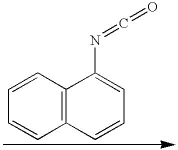 | 29-56 |
| 28 | 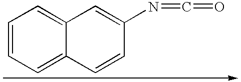 | 29-57 |
| 28 | 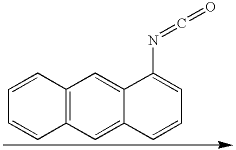 | 29-58 |
| 28 | 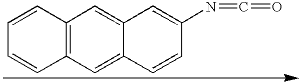 | 29-59 |
| 28 | 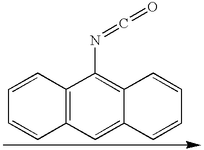 | 29-60 |

### Example 25

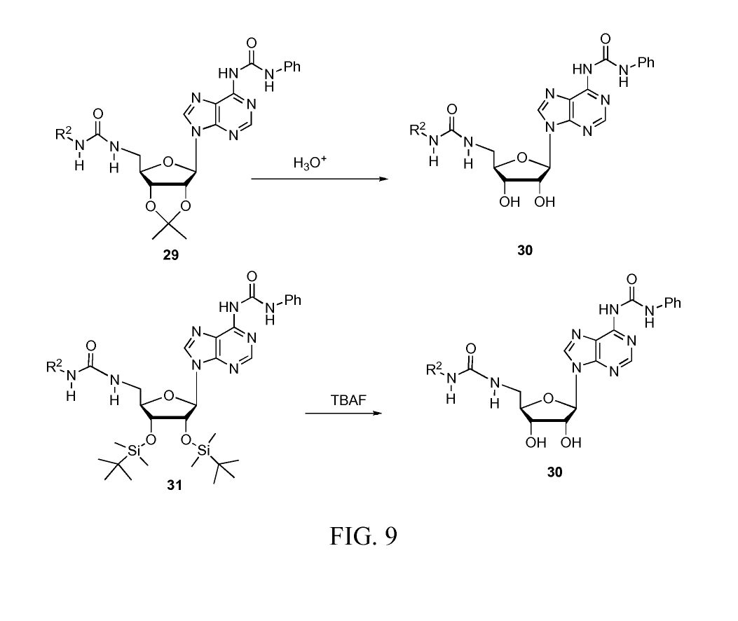

### Synthesis of 5′-Deoxy-5′-[(N-R²-substitutedcarbamoyl)amino]-N⁶-(N-phenylcarbamoyl)adenosine (Compound 30; FIG. 9)

Method A: A solution of Compound 29 and aqueous acid is vigorously stirred until TLC indicates complete conversion of Compound 29 to Compounds 30. Solvents are evaporated and the crude residue is chromatographed to give Compound 30.

Method B: A solution of Compound 31 and tetrabutylammonium fluoride (TBAF, 2.2 equiv.) in THF is stirred until TLC indicates complete conversion of starting material to Compound 30. Solvents are evaporated and the crude residue is chromatographed to give Compound 30.

### Examples 26-27

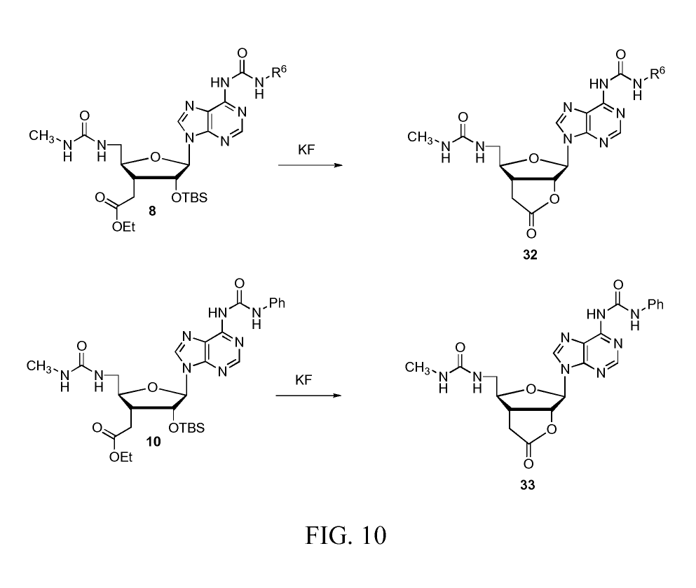

### Synthesis of Compounds 31-32 (FIG. 10)

### Example 26

### Synthesis of 3′-(Carboxymethyl)-3′,5′-dideoxy-5′-[(N-methylcarbamoyl)amino]-M-(N-R⁶-substitutedcarbamoyl)adenosine-2′,3′-lactone (Compound 32)

PhCH₂N(Et)₃Cl (1.7 equiv.), KF (3.0 equiv.), and H₂O are added to a stirred solution of Compound 8 in CH₃CN. The mixture is vigorously stirred at ambient temperature until TLC indicates that Compound 8 has been consumed. Silica gel is added and volatiles are evaporated under reduced pressure (≦20° C.). The dried silica gel is poured onto the top of a column packed with 5% MeOH/CH₂Cl₂ and eluted (5*10% MeOH/CH₂Cl₂). Evaporation of pooled fractions gives Compound 32.

### Example 27

### Synthesis of 3′-(Carboxymethyl)-3′,5′-dideoxy-5′-[(N-methylcarbamoyl)amino]-N⁶-(N-phenylcarbamoyl)adenosine-2′,3′-lactone (Compound 33)

PhCH₂N(Et)₃Cl (50 mg, 0.22 mmol), KF (22 mg, 0.38 mmol), and H₂O (80 μL) are added to a stirred solution of Compound 10 (82 mg, 0.131 mmol) in CH₃CN (3.0 mL). The mixture is vigorously stirred at ambient temperature until TLC indicates that Compound 10 had been consumed (60 h). Silica gel is added and volatiles are evaporated under reduced pressure (≦20° C.). The dried silica gel is poured onto the top of a column packed with 5% MeOH/CH₂Cl₂ and eluted (5*10% MeOH/CH₂Cl₂). Evaporation of pooled fractions gives Compound 33 (56 mg, 92%): UV (MeOH) λmax 279 nm (ε 23,200), λmin 240 nm; ¹H NMR (DMSO-d₆, 500 MHz) δ 11.74 (s, 1H), 10.18 (br s, 1H), 8.71 (s, 1H), 8.66 (s, 1H), 7.63 (d, J=8.0 Hz, 2H), 7.38-7.35 (m, 2H), 7.09 (t, J=7.5 Hz, 1H), 6.37 (d, J=2.0 Hz, 1H), 6.05 (t, J=6.0 Hz, 1H), 5.77 (dd, J=4.5, 8.5 Hz, 1H), 5.57 (dd, J=1.8, 7.3 Hz, 1H), 4.03-3.99 (m, 1H), 3.41-3.36 (m, 2H), 2.98 (dd, J=8.5, 18.0 Hz, 1H), 2.55 (d, J=5.0 Hz, 3H); ¹³C NMR (DMSO-d₆, 125 MHz) δ 176.3, 159.3, 151.8, 151.6, 150.8, 143.3, 139.2, 129.7, 123.9, 121.4, 120.1, 88.8, 87.5, 85.7, 42.4, 41.5, 40.7, 32.5, 27.1; MS (ES) m/z 467.1795 (MH⁺[C₂₁H₂₃N₈O₅]=467.1791).

### Examples 28-33

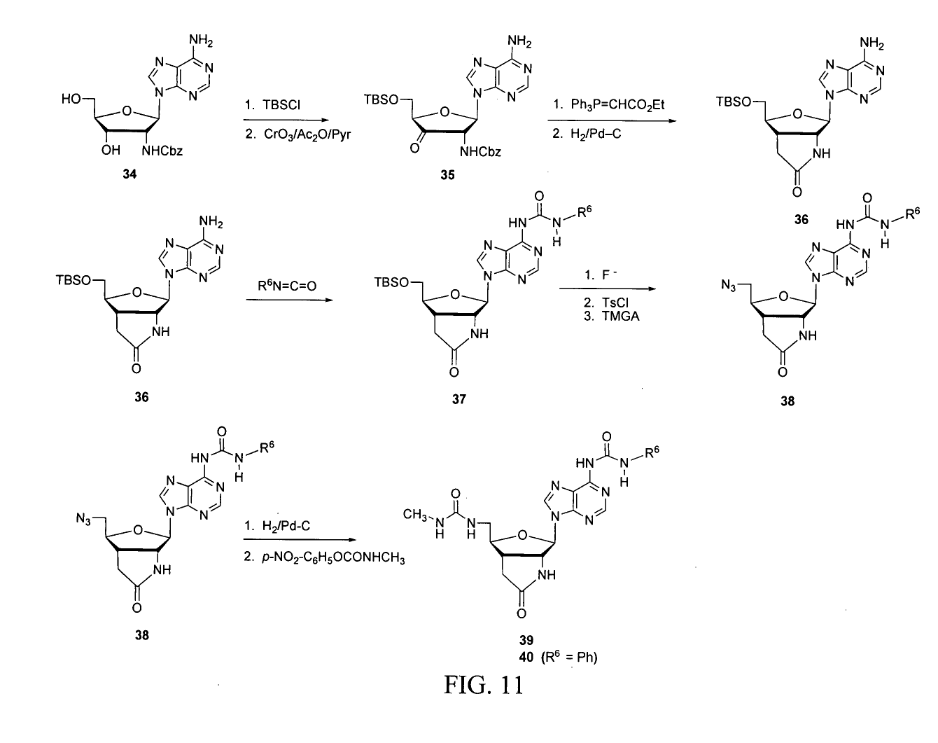

### Synthesis of Compounds 35-40 (FIG. 11)

### Example 28

### Synthesis of 5′-O-tert-Butyldimethylsilyl-2′-[(carbonylbenzyloxy)amino]-2′-deoxy-3′-ketoadenosine (Compound 35)

A solution of Compound 34 and tert-butyldimethylsilyl chloride (1.1 equiv.) in dry pyridine is stirred at ambient temperature until TLC indicates complete consumption of Compound 34. Volatiles are removed under reduced pressure and the residue is purified via column chromatography. The material thus obtained is dissolved in dry pyridine and treated with CrO₃/Ac₂O (2.0 equiv.) in pyridine for 2 h at ambient temperature. The mixture is poured into cold EtOAc (50-75 mL/mmol of Compound 34), the chromium salts are filtered through celite, and volatiles are removed under reduced pressure. The crude residue is chromatographed to give Compound 35.

### Example 29

### Synthesis of 5′-O-tert-Butyldimethylsilyl-3′-carboxymethyl-2′,3′-dideoxyadenosine-2′,3′-lactam (Compound 36)

A solution of Compound 35 and ethyl (triphenylphosphoranylidene)acetate (1.2 equiv.) in CH₂Cl₂ is refluxed overnight. Volatiles are removed under reduced pressure and the residue is chromatographed. The product thus obtained is dissolved in Ethanol and 10% Pd—C (1.5 equiv.; W/W) is added. The mixture is shaken under H₂ (60 psi) until TLC indicates complete conversion. The mixture is filtered (celite) and solvents are removed under reduced pressure. The crude reside is chromatographed to give Compound 36.

### Example 30

### Synthesis of 5′-O-tert-Butyldimethylsilyl-3′-carboxymethyl-2′,3′-dideoxy-N⁶-(N-R⁶-substitutedcarbamoyl)adenosine-2′,3′-lactam (Compound 37)

R⁶NCO (1.2 equiv.) is added to a stirred solution of Compound 36 in CH₂Cl₂. The mixture is stirred at ambient temperature until TLC indicates complete conversion of Compound 36 to Compound 37. The mixture is added directly to a chromatography column and eluted to give Compound 37.

### Example 31

### Synthesis of 5′-Azido-3′-carboxymethyl-2′,3′,5′-trideoxy-N⁶-(N-R⁶-substitutedcarbamoyl)adenosine-2′,3′-lactam (Compound 38)

A solution of Compound 37 and tetrabutylammonium fluoride (1.2 equiv.) is stirred at ambient temperature until TLC indicates complete cleavage of the tert-butyldimethylsilyl protecting group. Volatiles are removed under reduced pressure and the crude residue is chromatographed. The product thus obtained is treated with p-toluenesulphonylchloride (1.4 equiv.) and DMAP (2.1 equiv.) in ice-cold CH₂Cl₂. The solution is stirred for 24 h at 0° C., then applied directly to a chromatography column and eluted. Appropriate fractions are pooled and volatiles are removed under reduced pressure. The product thus obtained is treated with tetramethylguanidinium azide (TMGA, 7-10 equiv.) in DMF and the solution is heated at 65° C. for 7 h. The mixture is cooled to ambient temperature and then vigorously stirred while anhydrous Et₂O is slowly added. Precipitated tetramethylguanidinium azide is removed by filtering through celite. Volatiles are removed under reduced pressure and the residue is chromatographed to give Compound 38.

### Example 32

### Synthesis of 3′-Carboxymethyl-2′,3′,5′-trideoxy-5′-[(N-methylcarbamoyl)amino]-N⁶-(N-R⁶-substitutedcarbamoyl)adenosine-2′,3′-lactam (Compound 39)

A solution of Compound 38 and 10% Pd—C (306 mg/mmol Compound 38) in EtOAc is vigorously stirred for 15 h under an atmosphere of H₂ (balloon pressures). p-Nitrophenyl N-methylcarbamate (1.4 equiv.) and anhydrous Na₂CO₃ (2.5 equiv.) are added, and the resulting mixture is stirred for 4 h under N₂. Solids are removed via filtration (celite/EtOAc), and volatiles are evaporated under reduced pressure. The crude residue is chromatographed to give Compound 39.

### Example 33

### Synthesis of 3′-Carboxymethyl-2′,3′,5′-trideoxy-5′-[(N-methylcarbamoyl)amino]-N⁶-(N-phenylcarbamoyl)adenosine-2′,3′-lactam (Compound 40)

A solution of Compound 38 (R⁶=Ph) and 10% Pd—C (306 mg/mmol Compound 38) in EtOAc is vigorously stirred for 15 h under an atmosphere of H₂ (balloon pressures). p-Nitrophenyl N-methylcarbamate (1.4 equiv.) and anhydrous Na₂CO₃ (2.5 equiv.) are added, and the resulting mixture is stirred for 4 h under N₂. Solids are removed via filtration (celite/EtOAc), and volatiles are evaporated under reduced pressure. The crude residue is chromatographed to give Compound 40.

### Example 34

### Assay of Activity Change in Protein Kinase Targets in the Presence of Compound 10

The various protein kinase targets to be employed in the kinase profiling assay were cloned, expressed and purified in-house at SignalChem (Richmond, BC, Canada) using proprietary methods. Quality control testing is routinely performed on each of the SignalChem targets to ensure compliance to acceptable standards. Protein substrates employed in the target profiling process were synthesized internally. ³³P-ATP was purchased from PerkinElmer. All other materials were of standard grade. Compound 10 (FIG. 4) was supplied to SignalChem in a powder form. It was reconstituted in DMSO to form a stock solution which was then diluted with 10% DMSO to form a working stock solution (100 μM) that was then profiled against the various protein kinase targets. The assay conditions for the various protein kinase targets were optimized to yield acceptable enzymatic activity. In addition, the assays were optimized to give high signal-to-noise ratio.

Protein Kinase Assays

SignalChem uses a radioisotope assay format for profiling evaluation of protein kinase targets. Protein kinase assays were performed in triplicate at ambient temperature for 20-40 min (depending on the target) in a final volume of 25 μl according to the following assay reaction recipe:
Component 1: 5 μl of diluted active protein kinase target (˜10-40 nM final protein concentration in the assay)
Component 2: 5 μl of stock solution of substrate (1-5 μg of peptide or protein substrate)
Component 3: 5 μl of kinase assay buffer or protein kinase activator in kinase assay buffer
Component 4: 5 μl of Compound 10 (100 μM stock solution) or 10% DMSO
Component 5: 5 μl of 33P-ATP (25 μM stock solution, 0.8 μCi)

The assay was initiated by the addition of ³³P-ATP and the reaction mixture incubated at ambient temperature for 20-40 minutes, depending on the protein kinase target. After the incubation period, the assay was terminated by spotting 10 μl of the reaction mixture onto a Millipore Multiscreen plate. The Millipore Multiscreen plate was washed 3 times for approximately 15 minutes each in a 1% phosphoric acid solution. The radioactivity on the P81 plate was counted in the presence of scintillation fluid in a Trilux scintillation counter. Blank control, which included all the assay components except the addition of the appropriate substrate (replaced with equal volume of assay dilution buffer), was set up for each protein kinase target. The corrected activity for each protein kinase target was determined by removing the blank control value. Activity of the 52 kinase targets in the presence of Compound 10 (FIG. 4) is shown in Table 5. Activities for several of the target kinases were significantly enhanced, while two were markedly inhibited. These results demonstrate binding affinity of Compound 10 for several protein kinases.

TABLE 5
Change in protein kinase activity in the presence of Compound 10

| | % Activity |
|---|---|
| Target ID | Change |
| ABL1 | 0 |
| ABL2 | 8 |
| AKT1 | 2 |
| AKT2 | 27 |
| AKT3 | 52 |
| ALK4 | 2 |
| AURORA A | 1 |
| AURORA B | 4 |
| BRAF | 1 |
| BRK | 2 |
| BTK | 10 |
| c-KIT | −6 |
| ERK1 | 6 |
| ERK2 | −4 |
| FAK | −3 |
| FER | −3 |
| FGR | 3 |
| FLT3 | 11 |
| FMS | −17 |
| FRK | 24 |
| HCK | 18 |
| HER2 | 4 |
| KDR | 2 |
| LCK | 30 |
| LYN B | 12 |
| MARK1 | 5 |
| MARK3 | −1 |
| MEK1 | 8 |
| MST4 | 47 |
| NEK6 | 28 |
| p38α | 9 |
| p38β | 15 |
| _{P}38γ | 14 |
| p38δ | 5 |
| p70S6K | −1 |
| PAK2 | 16 |
| PAK3 | 524 |
| PAK4 | −20 |
| PAK7 | 21 |
| PDGFRα | −2 |
| PDGFRβ | −7 |
| PDK1 | 8 |
| PIM1 | 51 |
| PIM2 | 18 |
| PKCα | 36 |
| RAF1(EE) | 0 |
| RSK1 | 4 |
| RSK2 | −4 |
| RSK3 | 10 |
| SGK1 | 36 |
| SRC | −2 |
| TRKA | −6 |

### Example 35

### Inhibition of Binding of ATP-Binding-Site Ligands to Protein Kinases in the Presence of Compound 10

The novel binding affinity of Compound 10 (FIG. 4) for ATP-binding sites in protein kinases can be demonstrated by results from a kinase interaction assay performed by Ambit Biosciences, Inc. (San Diego, Calif., USA). This assay is based on ligand-affinity/protein kinase phage display and was employed essentially as described by Fabian et. al [Nature Biotech. 2005, 23, 329], which is incorporated herein by reference. In this assay, protein kinases are cloned into T7 bacteriophage which express the kinase fusion proteins on the phage capsid. T7 kinase-tagged phage are then screened for binding to ATP-binding-site ligands that have been immobilized on a solid support. Phage are screened for binding to the anchored ligands both in the presence of test compound and in its absence (control). Elution of the bound phage by free ligand (ATP-binding site ligand that is not immobilized on a solid support) followed by determination of the phage titre provides a reliable measure of the ability of test compounds to block binding of target kinases to resin-bound ATP-binding-site ligands. This method has allowed rapid mapping of small molecule interactions with ATP-binding sites across a broad cross-section of disease related protein kinases and has been validated as a reliable tool for identifying ligands with strong affinities for ATP-binding sites in numerous protein kinases (see Fabian et. al Nature Biotech. 2005, 23, 329).

Compound 10 (FIG. 4) was dissolved in DMSO to make a 1,000-X stock solution which was diluted to 10 μM in aqueous assay buffer system. T7 kinase-tagged phage strains were grown in parallel in microtiter plates in a proprietary bacterial host derived from E. Coli strain BL21. E. Coli were grown to a log phase, infected with T7 kinase-tagged phage, and incubated while shaking at 32° C. until bacterial lysis (approx. 90 min). Lysates were centrifuged (6,000 g) and filtered (0.2 μm). Small molecule ATP-binding-site-specific ligands were anchored to solid supports via a two step process beginning with biotin conjugation followed by treatment of the biotin/small molecule conjugate with streptavidin-coated magnetic beads. Derivatized beads were blocked by treatment with excess biotin followed by washing with blocking buffer (SeaBlock (Pierce), 1% BSA, 0.05% Tween 20, 1 mM DTT) to minimize nonspecific phage binding. Binding reactions were assembled by combining ATP-binding-site-ligand derivatized affinity beads, phage lysates, and Compound 10 (FIG. 4) in 1× assay buffer in polystyrene microtitreplates that had been pretreated with blocking buffer. Assay plates were incubated with shaking at 25° C. for 1 h. The beads were then washed with wash buffer (four times; 1×PBS, 0.05% Tween 20, 1 mM DTT) to remove unbound phage. The beads were then suspended in elution buffer (1×PBS, 0.05% Tween 20, 2 μM nonbiotinylated affinity ligand) and incubated with shaking for 30 min at 25° C. The phage titre of the eluates was measured by quantitative PCR or by plaque assays. Results were reported as percent inhibition of binding of phage to the resin-bound ATP-binding-site ligand. Compound 10 (FIG. 4) inhibited binding of 11 of the 353 protein kinases by ≧30% (Table 6). Kinases evaluated in this assay are shown in Table 7. ALK6 was inhibited by 47%. (ALK6 has recently been shown to play a key role in breast cancer tumorigenesis, see Breast Cancer Res Treat 2007, 103, 239-246). An additional 32 kinases were inhibited by 20-29%: TXK, RPS6KA2, MEK6, MAP4K5, EPHA5, CLK4, CIT, CD2 L2, ABL1(F317), SNF1LK, MLK1, ERK2, CLK3, MST1, MINK, KIT(D816V), EGFR(L747-T751del,Sins), CSF1R, CDK3, BMPR2, PIK3CG, HCK, RPS6KA6(kin.Dom.2), PHKG2, MET, AURKB, PDGFRB, DAPK1, CAMKK2, TYK2(Kin.Dom.1), p38-beta, CDK5.

TABLE 6
Inhibition of binding interactions between ATP-binding-site
ligands and protein kinases in the presence of Compound 10.

| Kinase | % Inhibition |
|---|---|
| EGFR | 30 |
| TYK2 | 31 |
| FLT3 | 31 |
| CSNK2A2 | 31 |
| PAK3 | 33 |
| MARK3 | 34 |
| BTK | 35 |
| IKK-α | 37 |
| CSNK1G2 | 38 |
| RPSGKA1 | 40 |
| ALK6 | 47 |

TABLE 7
List of protein kinases tested in the presence of compound 10.

AAK1, ABL1, ABL1(E255K), ABL1(F317I), ABL1(F317L), ABL1(H396P),
ABL1(M351T), ABL1 (Q252H), ABL1(T315I), ABL1(Y253F), ABL2, ACVR1, ACVR1B,
ACVR2A, ACVR2B, ACVRL1, ADCK3, ADCK4, AKT1, AKT2, AKT3, ALK, AMPK-
alpha1, AMPK-alpha2, ANKK1, ARK5, AURKA, AURKB, AURKC, AXL, BIKE, BLK,
BMPR1A, BMPR1B, BMPR2, BMX, BRAF, BRAF(V600E), BRSK1, BRSK2, BTK,
CAMK1, CAMK1D, CAMK1G, CAMK2A, CAMK2B, CAMK2D, CAMK2G,
CAMK4, CAMKK1, CAMKK2, CDC2L1, CDC2L2, CDK11, CDK2, CDK3, CDK5,
CDK7, CDK8, CDK9, CDKL2, CHEK1, CHEK2, CIT, CLK1, CLK2, CLK3, CLK4,
CSF1R, CSK, CSNK1A1L, CSNK1D, CSNK1E, CSNK1G1, CSNK1G2, CSNK1G3,
CSNK2A1, CSNK2A2, DAPK1, DAPK2, DAPK3, DCAMKL1, DCAMKL2, DCAMKL3,
DDR1, DDR2, DLK, DMPK, DMPK2, DRAK1, DRAK2, DYRK1B, EGFR, EGFR(E746-
A750del), EGFR(G719C), EGFR(G719S), EGFR(L747-E749del, A750P), EGFR(L747-
S752del, P753S), EGFR(L747-T751del, Sins), EGFR(L858R), EGFR(L861Q),
EGFR(S752-I759del), EPHA1, EPHA2, EPHA3, EPHA4, EPHA5, EPHA6, EPHA7,
EPHA8, EPHB1, EPHB2, EPHB3, EPHB4, ERBB2, ERBB4, ERK1, ERK2, ERK3, ERK4,
ERK5, ERK8, FER, FES, FGFR1, FGFR2, FGFR3, FGFR3(G697C), FGFR4, FGR, FLT1,
FLT3, FLT3(D835H), FLT3(D835Y), FLT3(ITD), FLT3(K663Q), FLT3(N841I), FLT4,
FRK, FYN, GAK, GCN2(Kin.Dom.2, S808G), GSK3A, GSK3B, HCK, HIPK1, 1GF1R,
IKK-alpha, IKK-beta, IKK-epsilon, INSR, INSRR, IRAK3, ITK, JAK1
(Kin.Dom.1), JAK1(Kin.Dom.2), JAK2(Kin.Dom.2), JAK3(Kin.Dom.2), JNK1, JNK2,
JNK3, KIT, KIT(D816V), KIT(V559D), KIT(V559D, T6701), K1T(V559D, V654A), LATS1,
LATS2, LCK, LIMK1, LIMK2, LKB1, LOK, LTK, LYN, MAP3K3, MAP3K4, MAP3K5,
MAP4K1, MAP4K2, MAP4K3, MAP4K4, MAP4K5, MAPKAPK2, MAPKAPK5,
MARK1, MARK2, MARK3, MARK4, MEK1, MEK2, MEK3, MEK4, MEK6, MELK,
MERTK, MET, MINK, MKNK1, MKNK2, MLCK, MLK1, MLK2, MLK3, MRCKA,
MRCKB, MST1, MST1R, MST2, MST3, MST4, MUSK, MYLK, MYLK2, MYO3A,
MYO3B, NDR2, NEK1, NEK2, NEK5, NEK6, NEK7, NEK9, NLK, p38-alpha, p38-beta,
p38-delta, p38-gamma, PAK1, PAK2, PAK3, PAK4, PAK6, PAK7/PAK5, PCTK1, PCTK2,
PCTK3, PDGFRA, PDGFRB, PDPK1, PFTAIRE2, PFTK1, PHKG1, PHKG2, PIK3C2B,
PIK3CA, PIK3CA(E545K), PIK3CB, PIK3CD, PIK3CG, PIM1, PIM2, PIM3, PIP5K1A,
PIP5K2B, PKAC-alpha, PKAC-beta, PKMYT1, PKN1, PKN2, PLK1, PLK3, PLK4,
PRKCD, PRKCE, PRKCH, PRKCQ, PRKD1, PRKD2, PRKD3, PRKG1, PRKG2,
PRKR, PRKX, PTK2, PTK2B, PTK6, RAF1, RET, RET(M918T), RET(V804L),
RET(V804M), RIOK1, RIOK2, RIOK3, RIPK1, RIPK2, RIPK4, ROCK2, ROS1,
RPS6KA1(Kin.Dom.1), RPS6KA1(Kin.Dom.2), RPS6KA2(Kin.Dom.1),
RPS6KA2(Kin.Dom.2), RPS6KA3(Kin.Dom.1), RPS6KA4 (Kin.Dom.1),
RPS6KA4(Kin.Dom.2), RPS6KA5(Kin.Dom.1), RPS6KA5(Kin.Dom.2), RPS6KA6
(Kin.Dom.1), RPS6KA6(Kin.Dom.2), SgK085, SgK110, SLK, SNARK, SNF1LK,
SNF1LK2, SRC, SRMS, SRPK1, SRPK2, SRPK3, STK16, STK33, STK35, STK36, SYK,
TAK1, TAOK1, TAOK3, TEC, TESK1, TGFBR1, TGFBR2, TIE1, TIE2, TLK1, TLK2,
TNIK, TNK1, TNK2, TNNI3K, TRKA, TRKB, TRKC, TSSK1, TTK, TXK,
TYK2(Kin.Dom.1), TYK2(Kin.Dom.2), TYRO3, ULK1, ULK2, ULK3, VEGFR2, WEE1,
WEE2, YANK2, YANK3, YES, YSK1, ZAK, ZAP70

### Example 36

### Cancer Data

The novel antitumor activities of the compounds of the present invention are demonstrated in the U.S. National Cancer Institute's (NCI) human tumor in vitro screens for Compound 10, Compound 13 (both in FIG. 4), and Compound 33 (FIG. 10). The antitumor data from these screens is shown in Tables 8, 9, and 10, respectively.

The In Vitro Cell Line Screening Project (IVCLSP) is a dedicated service provided through the Developmental Therapeutics Program of the NCl and utilizes 60 different human tumor cell lines (the NCI 60). The NCI 60 panel consists of leukemia and melanoma, and cancers of the breast, ovary, brain, lung, prostate, colon, and kidney. The NCI 60 screen is performed in two stages. The first stage consists of evaluation of the compounds against the 60 cell lines at a single dose (10 μM) and compounds meeting pre-defined criteria are then evaluated at 5 additional doses in the second stage as described below. Data in Tables 8 and 9 represent multi-dose screening results for Compound 10 and Compound 13, while data in Table 10 represent results from a single dose screen for Compound 33.

### Methodology of the NCI 60 In Vitro Cancer Screen

The human tumor cell lines of the NCI 60 screening panel are grown in RPMI 1640 medium containing 5% fetal bovine serum and 2 mM L-glutamine. Cells are inoculated into 96 well microtiter plates in 100 μl, with plating densities ranging from 5,000 to 40,000 cells/well depending on the doubling time of individual cell lines. After cell inoculation, the microtiter plates are incubated at 37° C., 5% CO₂, 95% air and 100% relative humidity for 24 h prior to addition of experimental compounds.

After 24 h, two plates of each cell line are fixed in situ with trichloroacetic acid (TCA), to obtain a measurement of the cell population for each cell line at the time of compound addition (Tz). Experimental compounds are solubilized in dimethyl sulfoxide at 400-X the desired final maximum test concentration and stored frozen prior to use. At the time of compound addition, an aliquot of frozen concentrate is thawed and diluted to 2-X the desired final maximum test concentration with complete medium containing 50 μg/ml gentamicin. Additional four, 10-fold or ½ log serial dilutions are made to provide a total of five compound concentrations plus control. Aliquots of 100 μl of these different compound dilutions are added to the appropriate microtiter wells already containing 100 μl of medium, resulting in the required final compound concentrations.

Following addition of the compound, the plates are incubated for an additional 48 h at 37° C., 5% CO₂, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (Tz), control growth, (C), and test growth in the presence of compound at the five concentration levels (Ti)], the percentage growth is calculated at each of the compound concentrations levels. Growth inhibition (GI) percentage is calculated as:

[(Ti−Tz)/(C−Tz)]×100 for concentrations for which Ti≧Tz

[(Ti−Tz)/Tz]×100 for concentrations for which Ti<Tz.

Three dose response parameters are calculated for each experimental agent. GI50 (the compound concentration required to inhibit cell growth by 50%) is calculated from [(Ti−Tz)/(C−Tz)]×100=50, and represents the compound concentration resulting in a 50% reduction in the net protein increase (as measured by SRB staining) in control cells during the compound incubation. TGI (the compound concentration resulting in total growth inhibition) is calculated from Ti=Tz. The LC50 (concentration of compound resulting in a 50% reduction in the measured protein at the end of compound treatment compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

GI50, TGI50, and LC50 for the Compounds are reported in Log 10 concentration values in Tables 8 and 9. Experimental data collected against each cell line is represented. The first column describes the subpanel (e.g. leukemia) and cell line (e.g. CCRF-CEM) involved, while the next two columns list the Mean OD_{tzero} and Mean OC_{ctr}. The next five columns list the Mean ODₜₑₛₜ for each of five different concentrations. Each concentration is expressed as the log₁₀ (molar). The next five columns list the calculated percent growth (PG) for each concentration. PG and Cl are equivalent terms with PG being used in Tables 8 and 9 and GI being used in Table 10. Definitions of OD terms for Tables 8 and 9 are as follows:

### Percentage Growth (PG)

The measured effect of the compound on a cell line is currently calculated according to one or the other of the following two expressions:

If(Mean ODₜₑₛₜ−Mean OD_{tzero})≧0.then [symbol] PG=100×(Mean ODₜₑₛₜ−Mean OD_{tzero})/(Mean ° D_{ctrl}−Mean OD_{tzero})

if(Mean ODₜₑₛₜ−Mean OD_{tzero})<0.then [symbol] PG=100×(Mean ODₜₑₛₜ−Mean OD_{tzero})/Mean OD_{tzero}

### Where:

Mean OD_{tzero}=The average of optical density measurements SRB-derived color just before exposure of cells to the test compound.
Mean ODₜₑₛₜ=The average of optical density measurement of SRB-derived color after 48 hours exposure of cells to the test compound.
Mean OD_{ctrl}=The average of optical density measurements of SRB-derived color after 48 hours with no exposure of cells to the test compound.

For Table 10, bars extending to the right represent sensitivity of cell line to the test agent in excess of the average sensitivity of all tested cell lines. Since the bar scale is logarithmic a bar 2 units to the right implies the compound achieved the response parameter (e.g. GI) for the cell line at a concentration one-hundredth the mean concentration required over all cell lines, and thus the cell line is usually sensitive to that compound. Bars extending to the left correspondingly imply sensitivity less than the mean.

Compound 33 shows potent and selective anticancer activities against the following cell lines (Table 10): Non Small Lung Cancer (HOP-92), Leukemia (MOLT-4), Renal Cancer (RXF393; UO-31), and Melanoma (LOX IMVI).

Compound 10 (FIG. 4) shows potent anticancer activities (low micromolar GI50 values) against the following cell lines (Table 8): Leukemia (CCRF-CEM; HL-60(TB); K-562; MOLT-4; RPMI-8226; SR); Non-Small Cell Lung Cancer (A549/ATCC; HOP-62; NCI-H460; NCI-H522); Colon Cancer (COLO 205; HCT-116; HCT-15; HT29; KM12; SW-620); CNS Cancer (SF-268; SF-295; SF-539; SNB-75; U251); Melanoma (LOX IMVI; M14; SK-MEL-2; SK-MEL-28; SK-MEL-5; UACC-257), Ovarian Cancer (IGROV1; OVCAR-3; OVCAR-8); Renal Cancer (786-0; A498; ACHN; RXF393; SN12C); Prostrate Cancer (PC-3, DU-145), and Breast Cancer (MCF7, MDA-MB-231/ATCC; HS578T; MDA-MB-435; T-47D). The LC50 value for each cell line was >100 micromolar.

Compound 13 (FIG. 4) shows potent anticancer activities (low micromolar GI50 values) against the following cell lines (Table 9): Leukemia (CCRF-CEM; HL-60(TB); K-562; MOLT-4; RPMI-8226; SR); Non-Small Cell Lung Cancer (A549/ATCC; HOP-92; NCI-H460); Colon Cancer (HCT-116; HCT-15; HT29); CNS Cancer (SF-268; SF-295; U251); Melanoma (LOX IMVI; SK-MEL-28; SK-MEL-5), Ovarian Cancer (IGROV1; OVCAR-3; OVCAR-8); Renal Cancer (A498; RXF393); and Breast Cancer (MCF7, HS578T). The LC50 value for each cell line was >100 micromolar.

TABLE 8
Antitumor activity of Compound 10.
National Cancer Institute Developmental Therapeutics Program
In-Vitro Testing Results

| | | | |
|---|---|---|---|
| NSC: 743565/1 | Experiment ID: 0707NS53 | Test Type: 08 | Units: Molar |
| Report Date: Aug. 23, 2007 | Test Date: Jul. 16, 2007 | QNS: | MC: |
| COMI: MAP-VII-102 (57361) | Stain Reagent: SRB Dual-Pass Related | SSPL: 0WPM | |

| | | | Log10 Concentration | | | | | | |
|---|---|---|---|---|---|---|---|---|---|
| | Time | | Mean Optical Densities | | | | | Percent Growth | |
| Panel/Cell Line | Zero | Ctrl | −8.0 | −7.0 | −6.0 | −5.0 | −4.0 | −8.0 | −7.0 |
| Leukemia | | | | | | | | | |
| CCRF-CEM | 0.765 | 2.522 | 2.444 | 2.521 | 2.387 | 1.487 | 0.748 | 96 | 100 |
| HL-60(TB) | 0.698 | 1.800 | 1.638 | 1.909 | 1.809 | 0.661 | 0.943 | 85 | 110 |
| K-562 | 0.297 | 1.576 | 1.524 | 1.485 | 1.439 | 0.535 | 0.246 | 96 | 93 |
| MOLT-4 | 0.503 | 1.956 | 1.853 | 1.719 | 1.603 | 0.618 | 0.435 | 93 | 84 |
| RPMI-8226 | 1.059 | 2.409 | 2.100 | 2.074 | 1.773 | 0.771 | 0.458 | 77 | 75 |
| SR | 0.806 | 1.388 | 1.284 | 1.212 | 1.299 | 0.685 | 0.528 | 82 | 70 |
| Non-Small Cell Lung Cancer | | | | | | | | | |
| A549/ATCC | 0.417 | 1.813 | 1.847 | 1.858 | 1.733 | 0.739 | 0.174 | 102 | 103 |
| EKVX | 1.065 | 2.314 | 2.215 | 2.190 | 2.204 | 1.724 | 1.587 | 92 | 90 |
| HOP-62 | 0.544 | 1.205 | 1.199 | 1.150 | 1.188 | 0.859 | 0.189 | 99 | 92 |
| HOP-92 | 0.982 | 1.226 | 0.991 | 0.975 | 1.011 | 0.664 | 0.383 | 4 | −1 |
| NCI-H226 | 0.943 | 1.982 | 1.996 | 2.037 | 2.007 | 1.685 | 1.513 | 101 | 105 |
| NCI-H23 | 0.598 | 1.778 | 1.788 | 1.784 | 1.784 | 1.371 | 1.022 | 101 | 100 |
| NCI-H322M | 0.664 | 1.870 | 1.935 | 1.744 | 1.814 | 1.737 | 1.531 | 105 | 90 |
| NCI-H460 | 0.274 | 1.811 | 1.844 | 1.841 | 1.827 | 0.772 | 0.140 | 102 | 102 |
| NCI-H522 | 0.527 | 1.537 | 1.425 | 1.386 | 1.362 | 0.847 | 0.515 | 89 | 85 |
| Colon Cancer | | | | | | | | | |
| COLO 205 | 0.243 | 0.938 | 0.952 | 0.916 | 0.887 | 0.380 | 0.267 | 102 | 97 |
| HCC-2998 | 0.878 | 2.513 | 2.532 | 2.440 | 2.405 | 2.010 | 1.888 | 101 | 96 |
| HCT-116 | 0.174 | 0.807 | 0.762 | 0.889 | 0.667 | 0.318 | 0.022 | 93 | 113 |
| HCT-15 | 0.258 | 1.821 | 1.799 | 1.755 | 1.732 | 0.987 | 0.929 | 99 | 96 |
| HT29 | 0.218 | 1.666 | 1.728 | 1.732 | 1.727 | 0.469 | 0.269 | 104 | 105 |
| KM12 | 0.294 | 0.920 | 0.963 | 0.993 | 0.951 | 0.375 | 0.213 | 107 | 112 |
| SW-620 | 0.124 | 0.741 | 0.721 | 0.707 | 0.726 | 0.296 | 0.083 | 97 | 94 |
| CNS Cancer | | | | | | | | | |
| SF-268 | 0.553 | 1.306 | 1.318 | 1.332 | 1.253 | 0.856 | 0.259 | 102 | 103 |
| SF-295 | 0.731 | 2.428 | 2.278 | 2.275 | 2.227 | 1.374 | 1.132 | 91 | 91 |
| SF-539 | 0.728 | 1.819 | 1.785 | 1.722 | 1.848 | 1.045 | 0.919 | 97 | 91 |
| SNB-19 | 0.449 | 1.372 | 1.294 | 1.324 | 1.254 | 1.210 | 0.562 | 92 | 95 |
| SNB-75 | 0.489 | 1.016 | 0.976 | 0.965 | 0.938 | 0.657 | 0.604 | 92 | 90 |
| U251 | 0.226 | 1.201 | 1.184 | 1.151 | 1.142 | 0.504 | 0.089 | 98 | 95 |
| Melanoma | | | | | | | | | |
| LOX IMVI | 0.398 | 2.417 | 2.364 | 2.347 | 2.276 | 1.098 | 0.476 | 97 | 97 |
| MALME-3M | 0.638 | 1.353 | 1.381 | 1.318 | 1.298 | 0.999 | 0.763 | 104 | 95 |
| M14 | 0.478 | 1.034 | 0.977 | 0.932 | 0.927 | 0.499 | 0.209 | 90 | 82 |
| SK-MEL-2 | 0.381 | 0.737 | 0.744 | 0.717 | 0.732 | 0.496 | 0.267 | 102 | 94 |
| SK-MEL-28 | 0.284 | 0.721 | 0.739 | 0.749 | 0.722 | 0.460 | 0.026 | 104 | 106 |
| SK-MEL-5 | 0.558 | 1.677 | 1.637 | 1.678 | 1.559 | 0.867 | 0.676 | 96 | 100 |
| UACC-257 | 0.894 | 1.564 | 1.581 | 1.576 | 1.558 | 1.122 | 0.956 | 102 | 102 |
| UACC-62 | 0.715 | 2.513 | 2.508 | 2.557 | 2.467 | 2.197 | 1.802 | 100 | 102 |
| Ovarian Cancer | | | | | | | | | |
| IGROV1 | 0.412 | 1.166 | 1.119 | 1.123 | 1.111 | 0.562 | 0.174 | 94 | 94 |
| OVCAR-3 | 0.367 | 0.725 | 0.775 | 0.778 | 0.784 | 0.425 | 0.174 | 114 | 115 |
| OVCAR-4 | 0.381 | 1.221 | 1.207 | 1.244 | 1.178 | 0.820 | 0.495 | 98 | 103 |
| OVCAR-5 | 0.465 | 1.129 | 1.153 | 1.147 | 1.098 | 0.944 | 0.646 | 104 | 103 |
| OVCAR-8 | 0.372 | 1.397 | 1.388 | 1.397 | 1.377 | 0.666 | 0.359 | 99 | 100 |
| SK-OV-3 | 0.428 | 1.029 | 1.003 | 1.004 | 0.971 | 0.826 | 0.523 | 96 | 96 |
| Renal Cancer | | | | | | | | | |
| 788-0 | 0.594 | 1.190 | 1.145 | 1.086 | 1.107 | 0.392 | 0.018 | 92 | 83 |
| A498 | 0.743 | 1.309 | 1.281 | 1.265 | 1.260 | 0.814 | 0.436 | 95 | 92 |
| ACHN | 0.435 | 1.672 | 1.764 | 1.631 | 1.624 | 1.012 | 0.866 | 107 | 97 |
| CAKI-1 | 0.700 | 1.005 | 0.969 | 0.990 | 1.002 | 0.970 | 0.722 | 88 | 95 |
| RXF 393 | 0.698 | 1.021 | 1.019 | 1.013 | 0.995 | 0.376 | 0.284 | 99 | 97 |
| SN12C | 0.479 | 1.665 | 1.673 | 1.681 | 1.699 | 1.045 | 0.615 | 101 | 101 |
| TK-10 | 0.562 | 1.213 | 1.199 | 1.235 | 1.245 | 0.946 | 0.348 | 98 | 103 |
| UO-31 | 0.467 | 1.206 | 1.108 | 1.111 | 1.070 | 0.918 | 0.146 | 87 | 87 |
| Prostate Cancer | | | | | | | | | |
| PC-3 | 0.208 | 0.411 | 0.453 | 0.463 | 0.417 | 0.110 | 0.049 | 121 | 126 |
| DU-145 | 0.275 | 0.717 | 0.763 | 0.818 | 0.763 | 0.380 | 0.114 | 110 | 123 |
| Breast Cancer | | | | | | | | | |
| MCF7 | 0.578 | 1.787 | 1.733 | 1.677 | 1.654 | 0.772 | 0.529 | 96 | 91 |
| NCI/ADR-RES | 0.544 | 1.705 | 1.767 | 1.787 | 1.746 | 1.476 | 1.226 | 105 | 107 |
| MDA-MB-231/ATCC | 0.390 | 0.903 | 0.908 | 0.921 | 0.874 | 0.494 | 0.341 | 101 | 103 |
| HS 578T | 0.506 | 1.037 | 1.008 | 1.010 | 0.983 | 0.604 | 0.472 | 94 | 95 |
| MDA-MB-435 | 0.516 | 1.977 | 1.906 | 1.861 | 1.820 | 1.097 | 0.630 | 95 | 92 |
| BT-549 | 0.913 | 1.929 | 1.972 | 2.024 | 2.026 | 1.593 | 1.427 | 104 | 109 |
| T-47D | 0.476 | 1.030 | 0.977 | 0.969 | 0.883 | 0.563 | 0.548 | 91 | 89 |

| | Log10 Concentration | | | | | |
|---|---|---|---|---|---|---|
| | Percent Growth | | | | | |
| Panel/Cell Line | −6.0 | −5.0 | −4.0 | GI50 | TGI | LC50 |
| Leukemia | | | | | | |
| CCRF-CEM | 92 | 41 | −2 | 6.69E−6 | 8.86E−5 | >1.00E−4 |
| HL-60(TB) | 101 | −5 | 22 | 3.01E−6 | | >1.00E−4 |
| K-562 | 89 | 19 | −17 | 3.59E−6 | 3.29E−5 | >1.00E−4 |
| MOLT-4 | 76 | 8 | −14 | 2.39E−6 | 2.33E−5 | >1.00E−4 |
| RPMI-8226 | 53 | −27 | −57 | 1.09E−6 | 4.57E−6 | 5.90E−5 |
| SR | 85 | −15 | −34 | 2.23E−6 | 7.07E−6 | >1.00E−4 |
| Non-Small Cell Lung Cancer | | | | | | |
| A549/ATCC | 94 | 23 | −58 | 4.18E−6 | 1.92E−5 | 7.91E−5 |
| EKVX | 91 | 53 | 42 | 1.77E−5 | >1.00E−4 | >1.00E−4 |
| HOP-62 | 97 | 48 | −65 | 8.96E−6 | 2.64E−5 | 7.31E−5 |
| HOP-92 | 12 | −32 | −61 | <1.00E−8 | . | 4.12E−5 |
| NCI-H226 | 102 | 71 | 55 | >1.00E−4 | >1.00E−4 | >1.00E−4 |
| NCI-H23 | 100 | 65 | 36 | 3.33E−5 | >1.00E−4 | >1.00E−4 |
| NCI-H322M | 95 | 89 | 72 | >1.00E−4 | >1.00E−4 | >1.00E−4 |
| NCI-H460 | 101 | 32 | −49 | 5.54E−6 | 2.50E−5 | >1.00E−4 |
| NCI-H522 | 83 | 32 | −2 | 4.36E−6 | 8.57E−5 | >1.00E−4 |
| Colon Cancer | | | | | | |
| COLO 205 | 93 | 20 | 3 | 3.84E−6 | >1.00E−4 | >1.00E−4 |
| HCC-2998 | 93 | 69 | 62 | >1.00E−4 | >1.00E−4 | >1.00E−4 |
| HCT-116 | 78 | 23 | −88 | 3.20E−6 | 1.61E−5 | 4.56E−5 |
| HCT-15 | 94 | 47 | 43 | 8.50E−6 | >1.00E−4 | >1.00E−4 |
| HT29 | 104 | 17 | 3 | 4.20E−6 | >1.00E−4 | >1.00E−4 |
| KM12 | 105 | 13 | −28 | 3.95E−6 | 2.09E−5 | >1.00E−4 |
| SW-620 | 97 | 28 | −33 | 4.80E−6 | 2.84E−5 | >1.00E−4 |
| CNS Cancer | | | | | | |
| SF-268 | 93 | 40 | −53 | 6.53E−6 | 2.70E−5 | 9.25E−5 |
| SF-295 | 88 | 38 | 24 | 5.73E−6 | >1.00E−4 | >1.00E−4 |
| SF-539 | 103 | 29 | 17 | 5.19E−6 | >1.00E−4 | >1.00E−4 |
| SNB-19 | 87 | 82 | 12 | 2.90E−5 | >1.00E−4 | >1.00E−4 |
| SNB-75 | 85 | 32 | 22 | 4.56E−6 | >1.00E−4 | >1.00E−4 |
| U251 | 94 | 28 | −61 | 4.69E−6 | 2.09E−5 | 7.60E−5 |
| Melanoma | | | | | | |
| LOX IMVI | 93 | 35 | 4 | 5.46E−6 | >1.00E−4 | >1.00E−4 |
| MALME-3M | 92 | 50 | 17 | 1.03E−5 | >1.00E−4 | >1.00E−4 |
| M14 | 81 | 4 | −56 | 2.51E−6 | 1.16E−5 | 7.86E−5 |
| SK-MEL-2 | 99 | 32 | −30 | 5.42E−6 | 3.31E−5 | >1.00E−4 |
| SK-MEL-28 | 100 | 40 | −91 | 6.85E−6 | 2.02E−5 | 4.87E−5 |
| SK-MEL-5 | 89 | 28 | 11 | 4.34E−6 | >1.00E−4 | >1.00E−4 |
| UACC-257 | 99 | 34 | 9 | 5.68E−6 | >1.00E−4 | >1.00E−4 |
| UACC-62 | 97 | 82 | 60 | >1.00E−4 | >1.00E−4 | >1.00E−4 |
| Ovarian Cancer | | | | | | |
| IGROV1 | 93 | 20 | −58 | 3.85E−6 | 1.80E−5 | 7.92E−5 |
| OVCAR-3 | 116 | 16 | −53 | 4.59E−6 | 1.72E−5 | 9.13E−5 |
| OVCAR-4 | 95 | 53 | 16 | 1.23E−5 | >1.00E−4 | >1.00E−4 |
| OVCAR-5 | 95 | 72 | 27 | 3.11E−5 | >1.00E−4 | >1.00E−4 |
| OVCAR-8 | 98 | 29 | −4 | 4.92E−6 | 7.72E−5 | >1.00E−4 |
| SK-OV-3 | 90 | 66 | 16 | 2.10E−5 | >1.00E−4 | >1.00E−4 |
| Renal Cancer | | | | | | |
| 788-0 | 86 | −34 | −97 | 2.00E−6 | 5.21E−8 | 1.79E−5 |
| A498 | 91 | 13 | −41 | 3.34E−6 | 1.71E−5 | >1.00E−4 |
| ACHN | 96 | 47 | 35 | 8.55E−6 | >1.00E−4 | >1.00E−4 |
| CAKI-1 | 99 | 88 | 7 | 2.97E−5 | >1.00E−4 | >1.00E−4 |
| RXF 393 | 92 | −46 | −59 | 2.01E−6 | 4.63E−6 | 1.95E−5 |
| SN12C | 103 | 48 | 11 | 9.10E−6 | >1.00E−4 | >1.00E−4 |
| TK-10 | 105 | 59 | −38 | 1.24E−5 | 4.05E−5 | >1.00E−4 |
| UO-31 | 82 | 61 | −69 | 1.21E−5 | 2.95E−5 | 7.17E−5 |
| Prostate Cancer | | | | | | |
| PC-3 | 103 | −47 | −77 | 2.25E−6 | 4.85E−6 | 1.25E−5 |
| DU-145 | 110 | 24 | −59 | 4.97E−6 | 1.94E−5 | 7.84E−5 |
| Breast Cancer | | | | | | |
| MCF7 | 89 | 16 | −8 | 3.42E−6 | 4.51E−5 | >1.00E−4 |
| NCI/ADR-RES | 104 | 80 | 59 | >1.00E−4 | >1.00E−4 | >1.00E−4 |
| MDA-MB-231/ATCC | 94 | 20 | −13 | 3.96E−6 | 4.13E−5 | >1.00E−4 |
| HS 578T | 90 | 18 | −7 | 3.60E−6 | 5.36E−5 | >1.00E−4 |
| MDA-MB-435 | 89 | 40 | 8 | 6.21E−6 | >1.00E−4 | >1.00E−4 |
| BT-549 | 110 | 67 | 51 | >1.00E−4 | >1.00E−4 | >1.00E−4 |
| T-47D | 73 | 16 | 13 | 2.55E−6 | >1.00E−4 | >1.00E−4 |

TABLE 9
Antitutmor activity of Compound 13.
National Cancer Institute Developmental Therapeutics Program
In-Vitro Testing Results

| | | | |
|---|---|---|---|
| NSC: 743564/1 | Experiment ID: 0707NS53 | Test Type: 08 | Units: Molar |
| Report Date: Aug. 23, 2007 | Test Date: Jul. 16, 2007 | QNS: | MC: |
| COMI: MAP-VII-54 (57360) | Stain Reagent: SRB Dual-Pass Related | SSPL: 0WPM | |

| | | | Log10 Concentration | | | | | | |
|---|---|---|---|---|---|---|---|---|---|
| | Time | | Mean Optical Densities | | | | | Percent Growth | |
| Panel/Cell Line | Zero | Ctrl | −8.0 | −7.0 | −6.0 | −5.0 | −4.0 | −8.0 | −7.0 |
| Leukemia | | | | | | | | | |
| CCRF-CEM | 0.765 | 2.408 | 2.426 | 2.416 | 2.344 | 1.403 | 1.147 | 101 | 100 |
| HL-60(TB) | 0.698 | 1.630 | 1.580 | 1.503 | 1.482 | 0.365 | 0.292 | 95 | 86 |
| K-562 | 0.297 | 1.291 | 1.244 | 1.243 | 1.112 | 0.468 | 0.534 | 95 | 95 |
| MOLT-4 | 0.503 | 1.762 | 1.708 | 1.639 | 1.484 | 0.492 | 0.479 | 96 | 90 |
| RPMI-8226 | 1.059 | 2.118 | 1.911 | 2.027 | 1.785 | 0.794 | 0.890 | 80 | 91 |
| SR | 0.806 | 1.328 | 1.248 | 1.268 | 1.129 | 0.380 | 0.354 | 85 | 89 |
| Non-Small Cell Lung Cancer | | | | | | | | | |
| A549/ATCC | 0.417 | 1.898 | 1.857 | 1.903 | 1.854 | 1.137 | 0.818 | 97 | 100 |
| EKVX | 1.065 | 2.611 | 2.516 | 2.536 | 2.536 | 1.993 | 1.627 | 94 | 95 |
| HOP-62 | 0.544 | 1.262 | 1.207 | 1.178 | 1.163 | 1.006 | 0.746 | 92 | 88 |
| HOP-92 | 0.982 | 1.233 | 1.182 | 1.216 | 1.151 | 1.050 | 0.556 | 80 | 93 |
| NCI-H226 | 0.943 | 2.011 | 2.039 | 2.133 | 2.002 | 1.645 | 1.376 | 103 | 111 |
| NCI-H23 | 0.598 | 1.704 | 1.771 | 1.736 | 1.693 | 1.382 | 1.077 | 106 | 103 |
| NCI-H322M | 0.664 | 1.908 | 1.869 | 1.886 | 1.933 | 1.789 | 1.571 | 97 | 98 |
| NCI-H460 | 0.274 | 1.835 | 1.776 | 1.726 | 1.674 | 0.966 | 0.669 | 96 | 93 |
| NCI-H522 | 0.527 | 1.448 | 1.355 | 1.324 | 1.297 | 0.996 | 0.814 | 90 | 87 |
| Colon Cancer | | | | | | | | | |
| COLO 205 | 0.243 | 1.050 | 1.027 | 0.992 | 0.968 | 0.685 | 0.251 | 97 | 93 |
| HCC-2998 | 0.878 | 2.590 | 2.630 | 2.536 | 2.593 | 2.005 | 1.447 | 102 | 97 |
| HCT-116 | 0.174 | 1.167 | 1.098 | 1.070 | 1.057 | 0.437 | 0.230 | 93 | 90 |
| HCT-15 | 0.258 | 1.930 | 1.868 | 1.827 | 1.812 | 0.927 | 0.875 | 96 | 94 |
| HT29 | 0.218 | 1.912 | 1.982 | 2.036 | 1.912 | 0.752 | 0.503 | 104 | 107 |
| KM12 | 0.294 | 1.018 | 1.023 | 1.079 | 1.063 | 0.743 | 0.513 | 101 | 108 |
| SW-620 | 0.124 | 0.737 | 0.729 | 0.752 | 0.693 | 0.525 | 0.433 | 99 | 102 |
| CNS Cancer | | | | | | | | | |
| SF-268 | 0.553 | 1.413 | 1.394 | 1.454 | 1.339 | 0.952 | 0.726 | 98 | 105 |
| SF-295 | 0.731 | 2.611 | 2.480 | 2.476 | 2.575 | 1.632 | 1.105 | 93 | 93 |
| SF-539 | 0.728 | 1.871 | 1.827 | 1.804 | 1.745 | 1.466 | 0.989 | 96 | 94 |
| SNB-19 | 0.449 | 1.426 | 1.367 | 1.354 | 1.265 | 1.048 | 0.950 | 94 | 93 |
| SNB-75 | 0.489 | 1.073 | 1.035 | 1.043 | 0.982 | 0.807 | 0.559 | 94 | 95 |
| U251 | 0.226 | 1.287 | 1.255 | 1.293 | 1.177 | 0.619 | 0.492 | 97 | 101 |
| Melanoma | | | | | | | | | |
| LOX IMVI | 0.398 | 2.549 | 2.486 | 2.473 | 2.415 | 1.324 | 1.146 | 97 | 96 |
| MALME-3M | 0.638 | 1.439 | 1.466 | 1.346 | 1.381 | 1.052 | 0.820 | 103 | 88 |
| M14 | 0.478 | 1.436 | 1.422 | 1.418 | 1.352 | 1.026 | 0.647 | 98 | 98 |
| SK-MEL-2 | 0.381 | 0.726 | 0.675 | 0.669 | 0.699 | 0.594 | 0.361 | 85 | 83 |
| SK-MEL-28 | 0.284 | 0.710 | 0.697 | 0.736 | 0.714 | 0.471 | 0.399 | 97 | 106 |
| SK-MEL-5 | 0.558 | 1.646 | 1.690 | 1.622 | 1.522 | 0.973 | 0.674 | 104 | 98 |
| UACC-257 | 0.894 | 1.767 | 1.724 | 1.795 | 1.784 | 1.462 | 1.090 | 95 | 103 |
| UACC-62 | 0.715 | 2.537 | 2.580 | 2.666 | 2.473 | 1.969 | 1.418 | 102 | 107 |
| Ovarian Cancer | | | | | | | | | |
| IGROV1 | 0.412 | 1.163 | 1.077 | 1.081 | 1.031 | 0.604 | 0.389 | 89 | 89 |
| OVCAR-3 | 0.367 | 0.781 | 0.760 | 0.808 | 0.780 | 0.538 | 0.393 | 95 | 107 |
| OVCAR-4 | 0.361 | 1.292 | 1.296 | 1.288 | 1.258 | 0.917 | 0.792 | 100 | 100 |
| OVCAR-5 | 0.465 | 1.106 | 1.098 | 1.092 | 1.053 | 0.921 | 0.688 | 99 | 98 |
| OVCAR-8 | 0.372 | 1.313 | 1.382 | 1.371 | 1.338 | 0.820 | 0.575 | 107 | 106 |
| SK-OV-3 | 0.428 | 1.010 | 0.989 | 0.995 | 0.984 | 0.905 | 0.648 | 96 | 97 |
| Renal Cancer | | | | | | | | | |
| 786-0 | 0.594 | 1.907 | 1.842 | 1.800 | 1.814 | 1.224 | 0.699 | 95 | 92 |
| A498 | 0.743 | 1.202 | 1.157 | 1.185 | 1.142 | 0.854 | 0.626 | 90 | 96 |
| ACHN | 0.435 | 1.791 | 1.892 | 1.816 | 1.726 | 1.182 | 0.746 | 107 | 102 |
| CAKI-1 | 0.700 | 1.046 | 0.993 | 0.949 | 0.964 | 0.897 | 0.864 | 85 | 72 |
| RXF 393 | 0.698 | 1.192 | 1.209 | 1.194 | 1.182 | 0.942 | 0.448 | 103 | 100 |
| SN12C | 0.479 | 1.852 | 1.929 | 1.902 | 1.795 | 1.380 | 1.150 | 106 | 104 |
| TK-10 | 0.562 | 1.186 | 1.177 | 1.223 | 1.209 | 0.968 | 0.667 | 99 | 106 |
| UO-31 | 0.467 | 1.343 | 1.246 | 1.246 | 1.183 | 0.871 | 0.752 | 89 | 89 |
| Prostate Cancer | | | | | | | | | |
| DU-145 | 0.275 | 0.783 | 0.821 | 0.842 | 0.802 | 0.578 | 0.357 | 107 | 112 |
| Breast Cancer | | | | | | | | | |
| MCF7 | 0.578 | 1.652 | 1.558 | 1.598 | 1.613 | 0.947 | 0.581 | 91 | 95 |
| NCI/ADR-RES | 0.544 | 1.622 | 1.717 | 1.717 | 1.675 | 1.361 | 1.139 | 109 | 109 |
| MDA-MB-231/ATCC | 0.390 | 0.966 | 0.947 | 0.990 | 0.933 | 0.704 | 0.417 | 97 | 104 |
| HS 578T | 0.506 | 0.999 | 0.992 | 0.989 | 0.912 | 0.703 | 0.567 | 98 | 98 |
| MDA-MB-435 | 0.516 | 2.016 | 2.023 | 1.969 | 1.987 | 1.275 | 1.049 | 100 | 97 |
| BT-549 | 0.913 | 1.990 | 1.940 | 1.965 | 1.946 | 1.671 | 1.197 | 95 | 98 |
| T-47D | 0.476 | 1.069 | 1.012 | 1.002 | 0.972 | 0.805 | 0.579 | 90 | 89 |

| | Log10 Concentration | | | | | |
|---|---|---|---|---|---|---|
| | Percent Growth | | | | | |
| Panel/Cell Line | −6.0 | −5.0 | −4.0 | GI50 | TGI | LC50 |
| Leukemia | | | | | | |
| CCRF-CEM | 96 | 39 | 23 | 6.37E−6 | >1.00E−4 | >1.00E−4 |
| HL-60(TB) | 84 | −48 | −58 | 1.81E−6 | 4.34E−6 | 1.64E−5 |
| K-562 | 82 | 17 | 24 | 3.12E−6 | >1.00E−4 | >1.00E−4 |
| MOLT-4 | 78 | −2 | −5 | 2.23E−6 | 9.36E−6 | >1.00E−4 |
| RPMI-8226 | 69 | −25 | −16 | 1.58E−6 | 5.40E−6 | >1.00E−4 |
| SR | 62 | −53 | −56 | 1.27E−6 | 3.46E−6 | 9.43E−6 |
| Non-Small Cell Lung Cancer | | | | | | |
| A549/ATCC | 97 | 49 | 27 | 9.35E−6 | >1.00E−4 | >1.00E−4 |
| EKVX | 95 | 60 | 36 | 2.64E−5 | >1.00E−4 | >1.00E−4 |
| HOP-62 | 86 | 64 | 28 | 2.49E−5 | >1.00E−4 | >1.00E−4 |
| HOP-92 | 67 | 27 | −43 | 2.71E−6 | 2.43E−5 | >1.00E−4 |
| NCI-H226 | 99 | 66 | 40 | 4.19E−5 | >1.00E−4 | >1.00E−4 |
| NCI-H23 | 99 | 71 | 43 | 5.72E−5 | >1.00E−4 | >1.00E−4 |
| NCI-H322M | 102 | 90 | 73 | >1.00E−4 | >1.00E−4 | >1.00E−4 |
| NCI-H460 | 90 | 44 | 25 | 7.49E−6 | >1.00E−4 | >1.00E−4 |
| NCI-H522 | 84 | 51 | 31 | 1.11E−5 | >1.00E−4 | >1.00E−4 |
| Colon Cancer | | | | | | |
| COLO 205 | 90 | 55 | 1 | 1.23E−5 | >1.00E−4 | >1.00E−4 |
| HCC-2998 | 100 | 66 | 33 | 3.06E−5 | >1.00E−4 | >1.00E−4 |
| HCT-116 | 89 | 26 | 6 | 4.20E−6 | >1.00E−4 | >1.00E−4 |
| HCT-15 | 93 | 40 | 37 | 6.47E−6 | >1.00E−4 | >1.00E−4 |
| HT29 | 100 | 32 | 17 | 5.37E−6 | >1.00E−4 | >1.00E−4 |
| KM12 | 106 | 62 | 30 | 2.39E−5 | >1.00E−4 | >1.00E−4 |
| SW-620 | 93 | 65 | 50 | >1.00E−4 | >1.00E−4 | >1.00E−4 |
| CNS Cancer | | | | | | |
| SF-268 | 91 | 46 | 20 | 8.29E−6 | >1.00E−4 | >1.00E−4 |
| SF-295 | 98 | 48 | 20 | 9.09E−6 | >1.00E−4 | >1.00E−4 |
| SF-539 | 89 | 65 | 23 | 2.23E−5 | >1.00E−4 | >1.00E−4 |
| SNB-19 | 83 | 61 | 51 | >1.00E−4 | >1.00E−4 | >1.00E−4 |
| SNB-75 | 84 | 54 | 12 | 1.27E−5 | >1.00E−4 | >1.00E−4 |
| U251 | 90 | 37 | 25 | 5.66E−6 | >1.00E−4 | >1.00E−4 |
| Melanoma | | | | | | |
| LOX IMVI | 94 | 43 | 35 | 7.30E−6 | >1.00E−4 | >1.00E−4 |
| MALME-3M | 93 | 52 | 23 | 1.14E−5 | >1.00E−4 | >1.00E−4 |
| M14 | 91 | 57 | 18 | 1.52E−5 | >1.00E−4 | >1.00E−4 |
| SK-MEL-2 | 92 | 62 | −5 | 1.49E−5 | 8.31E−5 | >1.00E−4 |
| SK-MEL-28 | 101 | 44 | 27 | 7.77E−6 | >1.00E−4 | >1.00E−4 |
| SK-MEL-5 | 89 | 38 | 11 | 5.81E−6 | >1.00E−4 | >1.00E−4 |
| UACC-257 | 102 | 65 | 22 | 2.26E−5 | >1.00E−4 | >1.00E−4 |
| UACC-62 | 96 | 69 | 39 | 4.19E−5 | >1.00E−4 | >1.00E−4 |
| Ovarian Cancer | | | | | | |
| IGROV1 | 82 | 26 | −6 | 3.72E−6 | 6.57E−5 | >1.00E−4 |
| OVCAR-3 | 100 | 41 | 6 | 7.11E−6 | >1.00E−4 | >1.00E−4 |
| OVCAR-4 | 96 | 60 | 46 | 5.30E−5 | >1.00E−4 | >1.00E−4 |
| OVCAR-5 | 92 | 71 | 35 | 3.82E−5 | >1.00E−4 | >1.00E−4 |
| OVCAR-8 | 103 | 48 | 22 | 9.02E−6 | >1.00E−4 | >1.00E−4 |
| SK-OV-3 | 95 | 82 | 38 | 5.27E−5 | >1.00E−4 | >1.00E−4 |
| Renal Cancer | | | | | | |
| 786-0 | 93 | 48 | 8 | 9.01E−6 | >1.00E−4 | >1.00E−4 |
| A498 | 87 | 24 | −16 | 3.87E−6 | 4.03E−5 | >1.00E−4 |
| ACHN | 95 | 55 | 23 | 1.44E−5 | >1.00E−4 | >1.00E−4 |
| CAKI-1 | 76 | 57 | 47 | 5.38E−5 | >1.00E−4 | >1.00E−4 |
| RXF 393 | 98 | 49 | −36 | 9.74E−6 | 3.80E−5 | >1.00E−4 |
| SN12C | 96 | 66 | 49 | 8.53E−5 | >1.00E−4 | >1.00E−4 |
| TK-10 | 104 | 65 | 17 | 2.05E−5 | >1.00E−4 | >1.00E−4 |
| UO-31 | 82 | 46 | 32 | 7.79E−6 | >1.00E−4 | >1.00E−4 |
| Prostate Cancer | | | | | | |
| DU-145 | 104 | 60 | 16 | 1.66E−5 | >1.00E−4 | >1.00E−4 |
| Breast Cancer | | | | | | |
| MCF7 | 96 | 34 | | 5.59E−6 | >1.00E−4 | >1.00E−4 |
| NCI/ADR-RES | 105 | 76 | 55 | >1.00E−4 | >1.00E−4 | >1.00E−4 |
| MDA-MB-231/ATCC | 94 | 55 | 5 | 1.23E−5 | >1.00E−4 | >1.00E−4 |
| HS 578T | 82 | 40 | 12 | 5.79E−6 | >1.00E−4 | >1.00E−4 |
| MDA-MB-435 | 98 | 51 | 36 | 1.09E−5 | >1.00E−4 | >1.00E−4 |
| BT-549 | 96 | 70 | 26 | 2.90E−5 | >1.00E−4 | >1.00E−4 |
| T-47D | 84 | 56 | 17 | 1.39E−5 | >1.00E−4 | >1.00E−4 |

TABLE 10
Anticancer data for Compound 33.

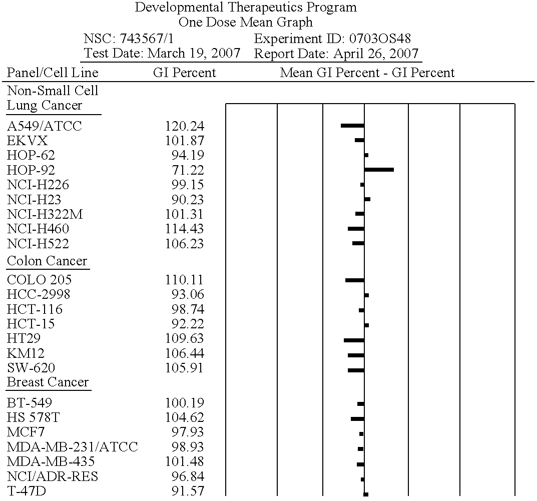

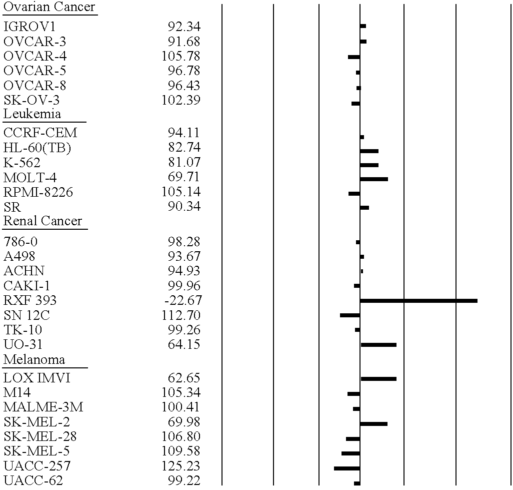

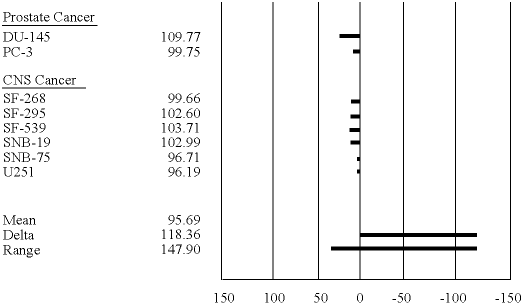

TABLE 11
Anticancer data for compound 25.
National Cancer Institute Developmental Therapeutics Program
In-Vitro Testing Results

| | | | |
|---|---|---|---|
| MSC: 750689/1 | Experiment ID: 0908NS02 | Test Type: 08 | Units: Molar |
| Report Date: Nov. 13, 2009 | Test Date: Aug. 03, 2009 | QNS: | MC: |
| COMI: MAP-VIII-70 (87585) | Slain Reagent: SRB Dual-Pass Related | SSPL: 0WPM | |

| | | | Log10 Concentration | | | | | | |
|---|---|---|---|---|---|---|---|---|---|
| | Time | | Mean Optical Densities | | | | | Percent Growth | |
| Panel/Cell Line | Zero | Ctrl | −8.0 | −7.0 | −6.0 | −5.0 | −4.0 | −8.0 | −7.0 |
| Leukemia | | | | | | | | | |
| CCRF-CEM | 0.342 | 1.591 | 1.633 | 1.700 | 1.603 | 0.458 | 0.394 | 104 | 109 |
| RPMI-8226 | 0.571 | 2.118 | 2.142 | 2.090 | 2.085 | 0.745 | 0.723 | 102 | 98 |
| Non-Small Cell Lung Cancer | | | | | | | | | |
| A549/ATCC | 0.376 | 1.448 | 1.435 | 1.432 | 1.339 | 0.415 | 0.262 | 99 | 98 |
| EKVX | 0.533 | 1.195 | 1.228 | 1.269 | 1.225 | 0.590 | 0.455 | 106 | 111 |
| HOP-52 | 0.511 | 1.355 | 1.385 | 1.430 | 1.409 | 0.734 | 0.074 | 102 | 108 |
| HOP-92 | 1.055 | 1.538 | 1.511 | 1.482 | 1.422 | 0.763 | 0.525 | 85 | 80 |
| NCI-H226 | 0.740 | 1.448 | 1.465 | 1.493 | 1.485 | 0.997 | 0.383 | 102 | 106 |
| NCI-H23 | 0.504 | 1.517 | 1.519 | 1.575 | 1.567 | 0.481 | 0.238 | 100 | 106 |
| NCI-H332M | 0.500 | 1.319 | 1.337 | 1.365 | 1.359 | 0.822 | 0.575 | 102 | 106 |
| NCI-H460 | 0.212 | 2.119 | 2.197 | 1.969 | 1.891 | 0.055 | 0.054 | 104 | 93 |
| NCI-H522 | 0.467 | 0.976 | 0.935 | 0.927 | 0.925 | 0.395 | 0.238 | 92 | 90 |
| Colon Cancer | | | | | | | | | |
| COLO 205 | 0.257 | 0.955 | 1.032 | 1.045 | 1.004 | 0.014 | 0.017 | 109 | 111 |
| HCC-2993 | 0.387 | 0.775 | 0.777 | 0.750 | 0.801 | 0.082 | 0.034 | 100 | 93 |
| HCT-116 | 0.202 | 1.358 | 1.429 | 1.413 | 1.374 | 0.083 | −0.004 | 107 | 105 |
| HCT-15 | 0.273 | 1.656 | 1.731 | 1.735 | 1.655 | 0.420 | 0.355 | 105 | 106 |
| HT29 | 0.255 | 1.223 | 1.243 | 1.265 | 1.291 | 0.051 | . | 102 | 104 |
| KM12 | 0.232 | 0.798 | 0.822 | 0.863 | 0.838 | 0.076 | 0.061 | 104 | 111 |
| CNS Cancer | | | | | | | | | |
| SF-268 | 0.439 | 1.252 | 1.237 | 1.297 | 1.272 | 0.502 | 0.242 | 104 | 106 |
| SF-295 | 0.417 | 1.119 | 1.143 | 1.163 | 1.195 | 0.465 | 0.399 | 103 | 107 |
| SF-539 | 0.438 | 1.383 | 1.831 | 1.832 | 1.758 | 0.533 | 0.213 | 100 | 100 |
| SNB-19 | 0.485 | 1.332 | 1.358 | 1.373 | 1.302 | 0.743 | 0.549 | 97 | 100 |
| SNB-75 | 0.657 | 1.050 | 1.028 | 1.031 | 0.943 | 0.711 | 0.578 | 94 | 95 |
| U251 | 0.258 | 1.316 | 1.335 | 1.340 | 1.245 | 0.276 | 0.141 | 102 | 102 |
| Melanoma | | | | | | | | | |
| LOX IMVI | 0.333 | 2.132 | 2.153 | 2.157 | 2.104 | 0.038 | 0.075 | 102 | 101 |
| MALME-3M | 0.602 | 1.169 | 1.199 | 1.169 | 1.145 | 0.598 | 0.311 | 105 | 88 |
| M14 | 0.406 | 1.252 | 1.257 | 1.311 | 1.291 | 0.566 | 0.195 | 101 | 107 |
| MDA-MB-435 | 0.407 | 1.496 | 1.520 | 1.523 | 1.463 | 0.502 | 0.358 | 102 | 102 |
| SK-MEL-2 | 0.505 | 0.972 | 1.011 | 1.019 | 0.995 | 0.322 | 0.168 | 106 | 110 |
| SK-MEL-5 | 0.447 | 2.154 | 2.188 | 2.118 | 1.964 | 0.256 | 0.005 | 102 | 96 |
| UACC-257 | 0.587 | 1.066 | 1.049 | 1.065 | 1.085 | 0.620 | 0.430 | 97 | 100 |
| UACC-62 | 0.647 | 2.365 | 2.391 | 2.365 | 2.195 | 1.047 | 0.394 | 102 | 100 |
| Ovarian Cancer | | | | | | | | | |
| IGROV1 | 0.436 | 1.105 | 1.107 | 1.098 | 1.096 | 0.383 | 0.264 | 100 | 99 |
| OVCAR-3 | 0.580 | 1.335 | 1.419 | 1.433 | 1.438 | 0.342 | 0.260 | 111 | 113 |
| OVCAR-4 | 0.533 | 1.184 | 1.220 | 1.238 | 1.182 | 0.579 | 0.512 | 106 | 106 |
| OVCAR-5 | 0.381 | 0.795 | 0.779 | 0.790 | 0.809 | 0.502 | 0.216 | 96 | 99 |
| OVCAR-8 | 0.406 | 1.283 | 1.358 | 1.335 | 1.334 | 0.523 | 0.371 | 108 | 106 |
| NCI/ADR-RES | 0.439 | 1.552 | 1.513 | 1.649 | 1.688 | 0.602 | 0.394 | 105 | 109 |
| SK-OV-3 | 0.401 | 0.931 | 0.976 | 1.001 | 0.571 | 0.607 | 0.442 | 106 | 113 |
| Renal Cancer | | | | | | | | | |
| 786-0 | 0.412 | 1.549 | 1.748 | 1.810 | 1.769 | 0.071 | 0.033 | 103 | 113 |
| A498 | 0.880 | 1.464 | 1.418 | 1.445 | 1.405 | 0.540 | 0.211 | 92 | 97 |
| ACHN | 0.368 | 1.552 | 1.632 | 1.653 | 1.617 | 0.491 | 0.395 | 107 | 108 |
| CAKI-1 | 0.582 | 1.691 | 1.727 | 1.678 | 1.646 | 0.520 | 0.392 | 100 | 99 |
| RXF 393 | 0.812 | 0.783 | 0.779 | 0.738 | 0.602 | 0.166 | 0.143 | 96 | 73 |
| SN12C | 0.479 | 1.883 | 1.878 | 1.888 | 1.851 | 0.061 | 0.050 | 100 | 100 |
| TK-10 | 0.492 | 1.269 | 1.350 | 1.370 | 1.383 | 0.600 | 0.481 | 110 | 113 |
| UO-31 | 0.479 | 1.055 | 0.955 | 1.049 | 0.972 | 0.471 | 0.380 | 87 | 97 |
| Prostate Cancer | | | | | | | | | |
| PC-3 | 0.368 | 1.530 | 1.443 | 1.429 | 1.332 | 0.440 | 0.412 | 93 | 91 |
| DU-145 | 0.292 | 0.938 | 0.960 | 0.983 | 0.999 | 0.189 | 0.031 | 103 | 107 |
| Breast Cancer | | | | | | | | | |
| MCF7 | 0.279 | 1.700 | 1.555 | 1.633 | 1.456 | 0.368 | 0.235 | 99 | 96 |
| MDA-MB-231/ATCC | 0.521 | 1.212 | 1.244 | 1.277 | 1.255 | 0.371 | 0.125 | 105 | 109 |
| BT-549 | 0.724 | 1.453 | 1.545 | 1.542 | 1.473 | 0.653 | 0.481 | 113 | 112 |
| T-47D | 0.387 | 0.819 | 0.822 | 0.842 | 0.783 | 0.389 | 0.252 | 101 | 105 |
| MDA-MB-468 | 0.594 | 1.401 | 1.407 | 1.435 | 1.392 | 0.642 | 0.388 | 101 | 104 |

| | Log10 Concentration | | | | | |
|---|---|---|---|---|---|---|
| | Percent Growth | | | | | |
| Panel/Cell Line | −6.0 | −5.0 | −4.0 | GI50 | TGI | IC50 |
| Leukemia | | | | | | |
| CCRF-CEM | 101 | 9 | 4 | 3.59E−6 | >1.00E−4 | >1.00E−4 |
| RPMI-8226 | 98 | 5 | 4 | 3.27E−6 | >1.00E−4 | >1.00E−4 |
| Non-Small Cell Lung Cancer | | | | | | |
| A549/ATCC | 90 | 4 | −30 | 2.90E−6 | 1.20E−5 | >1.00E−4 |
| EKVX | 104 | 9 | −9 | 3.70E−6 | 3.06E−5 | >1.00E−4 |
| HOP-52 | 105 | 25 | −55 | 4.98E−5 | 1.71E−5 | 4.90E−5 |
| HOP-92 | 69 | −25 | −50 | 1.55E−5 | 5.16E−6 | 9.75E−5 |
| NCI-H226 | 105 | −7 | −48 | 3.10E−6 | 3.63E−6 | >1.00E−4 |
| NCI-H23 | 105 | −5 | −53 | 3.17E−6 | 9.07E−6 | 8.50E−5 |
| NCI-H332M | 105 | 39 | 21 | 6.86E−6 | >1.00E−4 | >1.00E−4 |
| NCI-H460 | 88 | −74 | −75 | 1.72E−6 | 3.49E−6 | 7.11E−6 |
| NCI-H522 | 90 | −15 | −49 | 2.39E−6 | 7.14E−6 | >1.00E−4 |
| Colon Cancer | | | | | | |
| COLO 205 | 105 | −95 | −94 | 1.59E−6 | 3.35E−6 | 5.98E−6 |
| HCC-2993 | 106 | −79 | −91 | 2.02E−6 | 3.75E−6 | 6.99E−6 |
| HCT-116 | 102 | −59 | −100 | 2.11E−6 | 4.31E−6 | 8.80E−6 |
| HCT-15 | 100 | 11 | 6 | 3.62E−6 | >1.00E−4 | >1.00E−4 |
| HT29 | 107 | −80 | −100 | 2.02E−6 | 3.73E−6 | 8.91E−6 |
| KM12 | 107 | −66 | −74 | 2.13E−6 | 4.41E−6 | 8.05E−6 |
| CNS Cancer | | | | | | |
| SF-268 | 102 | 8 | 45 | 3.58E−6 | 1.40E−5 | >1.00E−4 |
| SF-295 | 111 | 7 | −7 | 3.84E−6 | 3.18E−5 | >1.00E−4 |
| SF-539 | 91 | 7 | −51 | 3.07E−6 | 1.30E−5 | 9.43E−5 |
| SNB-19 | 91 | 29 | 7 | 4.57E−6 | >1.00E−4 | >1.00E−4 |
| SNB-75 | 73 | 14 | −12 | 2.43E−6 | 3.41E−5 | >1.00E−4 |
| U251 | 93 | 2 | −46 | 2.96E−6 | 1.08E−5 | >1.00E−4 |
| Melanoma | | | | | | |
| LOX IMVI | 98 | −89 | −77 | 1.82E−6 | 3.36E−6 | 6.21E−5 |
| MALME-3M | 96 | −1 | −48 | 2.98E−6 | 9.84E−6 | >1.00E−4 |
| M14 | 105 | 19 | −52 | 4.33E−6 | 1.85E−5 | 9.38E−5 |
| MDA-MB-435 | 97 | 9 | −10 | 3.41E−6 | 2.91E−5 | >1.00E−4 |
| SK-MEL-2 | 105 | −35 | −67 | 2.45E−6 | 5.54E−6 | 2.83E−5 |
| SK-MEL-5 | 82 | −42 | −99 | 1.98E−6 | 4.75E−6 | 1.36E−5 |
| UACC-257 | 104 | 11 | −24 | 3.78E−6 | 2.02E−5 | >1.00E−4 |
| UACC-62 | 90 | 23 | −39 | 3.98E−6 | 2.36E−5 | >1.00E−4 |
| Ovarian Cancer | | | | | | |
| IGROV1 | 98 | −12 | −39 | 2.74E−6 | 7.76E−6 | >1.00E−4 |
| OVCAR-3 | 113 | −39 | −50 | 2.60E−6 | 5.54E−6 | 9.82E−5 |
| OVCAR-4 | 100 | 7 | −4 | 3.44E−6 | 4.39E−5 | >1.00E−4 |
| OVCAR-5 | 103 | 29 | −43 | 5.24E−6 | 2.52E−5 | >1.00E−4 |
| OVCAR-8 | 106 | 13 | −9 | 4.01E−6 | 4.01E−5 | >1.00E−4 |
| NCI/ADR-RES | 103 | 10 | −21 | 3.72E−6 | 2.07E−5 | >1.00E−4 |
| SK-OV-3 | 107 | 39 | 5 | 5.88E−6 | >1.00E−4 | >1.00E−4 |
| Renal Cancer | | | | | | |
| 786-0 | 110 | −83 | −80 | 2.04E−6 | 3.71E−6 | 6.75E−6 |
| A498 | 90 | −39 | −75 | 2.03E−6 | 4.06E−6 | 1.95E−6 |
| ACHN | 105 | 10 | 2 | 3.83E−6 | >1.00E−4 | >1.00E−4 |
| CAKI-1 | 96 | −11 | −33 | 2.70E−6 | 7.93E−6 | >1.00E−4 |
| RXF 393 | 111 | −73 | −77 | 2.14E−6 | 4.01E−6 | 7.50E−6 |
| SN12C | 98 | −87 | −90 | 1.81E−6 | 3.38E−6 | 6.29E−6 |
| TK-10 | 112 | 14 | −2 | 4.29E−6 | 7.26E−5 | >1.00E−4 |
| UO-31 | 84 | −2 | −21 | 2.50E−6 | 9.56E−6 | >1.00E−4 |
| Prostate Cancer | | | | | | |
| PC-3 | 83 | 6 | 4 | 2.69E−6 | >1.00E−4 | >1.00E−4 |
| DU-145 | 109 | −35 | −89 | 2.59E−6 | 5.71E−6 | 1.87E−5 |
| Breast Cancer | | | | | | |
| MCF7 | 83 | 5 | −15 | 2.69E−6 | 1.93E−5 | >1.00E−4 |
| MDA-MB-231/ATCC | 106 | −20 | −76 | 2.81E−6 | 5.12E−6 | 2.83E−5 |
| BT-549 | 103 | −10 | −35 | 2.92E−6 | 9.18E−6 | >1.00E−4 |
| T-47D | 92 | . | −32 | 2.87E−6 | 1.03E−5 | >1.00E−4 |
| MDA-MB-468 | 99 | 5 | −35 | 3.35E−6 | 1.39E−5 | >1.00E−4 |

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A molecule having the structure 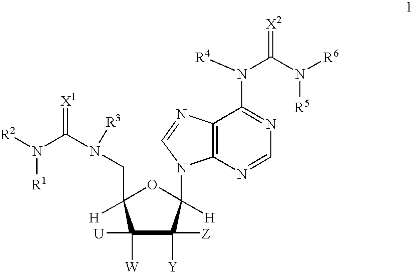
wherein:
R¹, R², R⁵, and R⁶, are members selected independently from the group consisting of H, HO—, CH₃O—, CH₃—, HOCH₂CH₂—, HOCH₂CH₂OCH₂CH₂—, NH₂CH₂CH₂—, R⁷NHCH₂CH₂—, (R⁷)₂NCH₂CH₂—, NH₂CH₂CH₂NHCH₂CH₂—, R⁷NHCH₂CH₂NHCH₂CH₂—, (R⁷)₂NCH₂CH₂NHCH₂CH₂—, R⁸CO—, a mono-, di-, or tri-cyclic aryl from C₆ to C₁₄, a mono-, di-, or tri-cyclic aryl from C₆ to C₁₄ mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂;
R⁷ is an alkyl from C₁ to C₅;
R⁸ is H₂N—, HOHN—, alkyl from C₁ to C₁₀, alkenyl from C₂ to C₁₀, or phenyl;
R⁹ is alkyl from C₁ to C₂₀;
R³ and R⁴ are members selected independently from the group consisting of H, HO—, CH₃—, or CH₃CH₂—;
X¹ and X² are members selected independently from the group consisting of O and S;
U is a member selected from the group consisting of H, HO—, F, CF₃—;
W is a member selected from the group consisting of H, HO—, F, CF₃—, CH₃CH₂O₂CCH₂—, CH₃(CH₃O)NCOCH₂—, HOCH₂CH₂O—, NH₂COCH₂—, CH₃NHCOCH₂—, (CH₃)₂NCOCH₂—, HOCH₂CH₂NHCOCH₂—, HSCH₂CH₂NHCOCH₂—, R⁹O—, and an O-trialkylsilyl containing three to sixteen carbons;
Y is a member selected from the group consisting of H, HO—, F, CF₃—, HOCH₂CH₂O—, R⁹O—, and an O-trialkylsilyl containing three to sixteen carbons; and
Z is a member selected from the group consisting of H, F, HO—, CF₃—, and R⁹O—.

2. The molecule of claim 1, wherein R¹ is H, R² is CH₃, R³ is H, R⁴ is H, R⁵ is H, U is H, W is CH₃CH₂O₂CCH₂—, Z is H, Y is O-tert-butyldimethylsilyl, X¹ is O, X² is O, and R⁶ is a member selected independently from the group consisting of a mono-, di-, or tri-cyclic aryl from C₆ to C₁₄, a mono-, di-, or tri-cyclic aryl from C₆ to C₁₄ mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; and
R⁹ is alkyl from C₁ to C₁₂.

3. The molecule of claim 2, wherein R⁶ is phenyl.

4. The molecule of claim 2, wherein R⁶ is a member selected independently from the group consisting of a mono-, di-, or tri-cyclic aryl from C₆ to C₁₄.

5. The molecule of claim 2, wherein R⁶ is a member selected independently from the group consisting of a mono-, di-, or tri-cyclic aryl from C₆ to C₁₄ mono-, di-, tri-, or poly-substituted with a member selected independently from the group consisting of F, Cl, Br, or I.

6. The molecule of claim 2, wherein R⁶ is a member selected independently from the group consisting of a mono-, di-, or tri-cyclic aryl from C₆ to C₁₄ mono-, di-, tri-, or poly-substituted with alkoxy (R⁹O—); and
R⁹ is alkyl from C₁ to C₁₂.

7. The molecule of claim 2, wherein R⁶ is a member selected independently from the group consisting of a mono-, di-, or tri-cyclic aryl from C₆ to C₁₄ mono-, di-, tri-, or poly-substituted with nitro (NO₂), nitroso (NO), or azido (N₃).

8. The molecule of claim 2, wherein R⁶ is a member selected independently from the group consisting of a mono-, di-, or tri-cyclic aryl from C₆ to C₁₄ mono-, di-, tri-, or poly-substituted with alkyl from C₂ to C₁₂, alkenyl from C₂ to C₁₂, alkynyl from C₂ to C₁₂, or acyl from C₂ to C₁₂.

9. The molecule of claim 2, wherein R⁶ is an O, N, or S mono- or bi-cyclic heterocycle having from two to nine carbon atoms.

10. The molecule of claim 2, wherein R⁶ is an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; and
R⁹ is alkyl from C₁ to C₁₂.

11. A molecule of claim 1 wherein R¹ is H, R² is CH₃, R³ is H, R⁴ is H, R⁵ is H, U is H, W is CH₃(CH₃O)NCOCH₂—, Z is H, Y is O-tert-butyldimethylsilyl, X¹ is O, X² is O, and R⁶ is phenyl.

12. A molecule of claim 1 wherein R¹ is H, R² is CH₃, R³ is H, R⁴ is H, R⁵ is H, U is H, W is OH, Z is H, Y is OH, X¹ is O, X² is O; and R⁶ is a member selected independently from the group consisting of a mono-, di-, or tri-cyclic aryl from C₆ to C₁₄, a mono-, di-, or tri-cyclic aryl from C₆ to C₁₄ mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂;
and wherein R⁹ is alkyl from C₁ to C₁₂.

13. A molecule of claim 12 wherein R⁶ is phenyl.

14. A molecule of claim 12 wherein R⁶ is a member selected independently from the group consisting of a mono-, di-, or tri-cyclic aryl from C₆ to C₁₄.

15. A molecule of claim 12 wherein R⁶ is a member selected independently from the group consisting of a mono-, di-, or tri-cyclic aryl from C₆ to C₁₄ mono-, di-, tri-, or poly-substituted with a member selected independently from the group consisting of F, Cl, Br, or I.

16. A molecule of claim 12 wherein R⁶ is a member selected independently from the group consisting of a mono-, di-, or tri-cyclic aryl from C₆ to C₁₄ mono-, di-, tri-, or poly-substituted with alkoxy (R⁹O—);
and wherein R⁹ is alkyl from C₁ to C₁₂.

17. A molecule of claim 12 wherein R⁶ is a member selected independently from the group consisting of a mono-, di-, or tri-cyclic aryl from C₆ to C₁₄ mono-, di-, tri-, or poly-substituted with nitro (NO₂), nitroso (NO), or azido (N₃).

18. A molecule of claim 12 wherein R⁶ is a member selected independently from the group consisting of a mono-, di-, or tri-cyclic aryl from C₆ to C₁₄ mono-, di-, tri-, or poly-substituted with alkyl from C₂ to C₁₂, alkenyl from C₂ to C₁₂, alkynyl from C₂ to C₁₂, or acyl from C₂ to C₁₂.

19. A molecule of claim 12 wherein R⁶ is an O, N, or S mono- or bi-cyclic heterocycle having from two to nine carbon atoms.

20. A molecule of claim 12 wherein R⁶ is an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂;
and wherein R⁹ is alkyl from C₁ to C₁₂.

21. A molecule of claim 1 wherein R¹ is H, R² is CH₃, R³ is H, R⁴ is H, R⁵ is H, U is H, Z is H, W and Y are —OC(CH₃)₂O—, X¹ is O, X² is O, and R⁶ is a member selected independently from the group consisting of a mono-, di-, or tri-cyclic aryl from C₆ to C₁₄, a mono-, di-, or tri-cyclic aryl from C₆ to C₁₄ mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂;
and wherein R⁹ is alkyl from C₁ to C₁₂.

22. A molecule of claim 21 wherein R⁶ is phenyl.

23. A molecule of claim 21 wherein R⁶ is a member selected independently from the group consisting of a mono-, di-, or tri-cyclic aryl from C₆ to C₁₄.

24. A molecule of claim 21 wherein R⁶ is a member selected independently from the group consisting of a mono-, di-, or tri-cyclic aryl from C₆ to C₁₄ mono-, di-, tri-, or poly-substituted with a member selected independently from the group consisting of F, Cl, Br, or I.

25. A molecule of claim 21 wherein R⁶ is a member selected independently from the group consisting of a mono-, di-, or tri-cyclic aryl from C₆ to C₁₄ mono-, di-, tri-, or poly-substituted with alkoxy (R⁹O—);
and wherein R⁹ is alkyl from C₁ to C₁₂.

26. A molecule of claim 21 wherein R⁶ is a member selected independently from the group consisting of a mono-, di-, or tri-cyclic aryl from C₆ to C₁₄ mono-, di-, tri-, or poly-substituted with nitro (NO₂), nitroso (NO), or azido (N₃).

27. A molecule of claim 21 wherein R⁶ is a member selected independently from the group consisting of a mono-, di-, or tri-cyclic aryl from C₆ to C₁₄ mono-, di-, tri-, or poly-substituted with alkyl from C₂ to C₁₂, alkenyl from C₂ to C₁₂, alkynyl from C₂ to C₁₂, or acyl from C₂ to C₁₂.

28. A molecule of claim 21 wherein R⁶ is an O, N, or S mono- or bi-cyclic heterocycle having from two to nine carbon atoms.

29. A molecule of claim 21 wherein R⁶ is an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂;
and wherein R⁹ is alkyl from C₁ to C₁₂.

30. A molecule of claim 1 wherein R¹ is H, R² is CH₃, R³ is H, R⁴ is H, R⁵ is H, U is H, Z is H, W is O-tert-butyldimethylsilyl, Y is O-tert-butyldimethylsilyl, X¹ is O, X² is O, and R⁶ is a member selected independently from the group consisting of a mono-, di-, or tri-cyclic aryl from C₆ to C₁₄, a mono-, di-, or tri-cyclic aryl from C₆ to C₁₄ mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂;
and wherein R⁹ is alkyl from C₁ to C₁₂.

31. A molecule of claim 30 wherein R⁶ is phenyl.

32. A molecule of claim 30 wherein R⁶ is a member selected independently from the group consisting of a mono-, di-, or tri-cyclic aryl from C₆ to C₁₄.

33. A molecule of claim 30 wherein R⁶ is a member selected independently from the group consisting of a mono-, di-, or tri-cyclic aryl from C₆ to C₁₄ mono-, di-, Irk or poly-substituted with a member selected independently from the group consisting of F, Cl, Br, or I.

34. A molecule of claim 30 wherein R⁶ is a member selected independently from the group consisting of a mono-, di-, or tri-cyclic aryl from C₆ to C₁₄ mono-, di-, tri-, or poly-substituted with alkoxy (R⁹O—);
and wherein R⁹ is alkyl from C₁ to C₁₂.

35. A molecule of claim 30 wherein R⁶ is a member selected independently from the group consisting of a mono-, di-, or tri-cyclic aryl from C₆ to C₁₄ mono-, di-, tri-, or poly-substituted with nitro (NO₂), nitroso (NO), or azido (N₃).

36. A molecule of claim 30 wherein R⁶ is a member selected independently from the group consisting of a mono-, di-, or tri-cyclic aryl from C₆ to C₁₄ mono-, di-, tri-, or poly-substituted with alkyl from C₂ to C₁₂, alkenyl from C₂ to C₁₂, alkynyl from C₂ to C₁₂, or acyl from C₂ to C₁₂.

37. A molecule of claim 30 wherein R⁶ is an O, N, or S mono- or bi-cyclic heterocycle having from two to nine carbon atoms.

38. A molecule of claim 30 wherein R⁶ is an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂;
and wherein R⁹ is alkyl from C₁ to C₁₂.

39. A molecule of claim 1 wherein R¹ is H, R³ is H, R⁴ is H, R⁵ is H, R⁶ is C₆H₅, U is H, W is CH₃CH₂O₂CCH₂—, Z is H, Y is O-tert-butyldimethylsilyl, X¹ is O, X² is O, and R² is selected independently from the group consisting of H, HO—, CH₃O—, CH₃—, HOCH₂CH₂—, HOCH₂CH₂OCH₂CH₂—, NH₂CH₂CH₂—, R⁷NHCH₂CH₂—, (R⁷)₂NCH₂CH₂—, NH₂CH₂CH₂NHCH₂CH₂—, R⁷NHCH₂CH₂NHCH₂CH₂—, (R⁷)₂NCH₂CH₂NHCH₂CH₂—, R⁸CO—, or a mono-, di-, or tri-cyclic aryl from C₆ to C₁₄;
and wherein R⁷ is an alkyl from C₁ to C₅; and R⁸ is H₂N—, HOHN—, alkyl from C₁ to C₁₀, alkenyl from C₂ to C₁₀, or phenyl.

40. A molecule of claim 1 wherein R¹ is H, R³ is H, R⁴ is H, R⁵ is H, R⁶ is C₆H₅, U is H, W is OH, Z is H, Y is OH, X¹ is O, X² is O, and R² is selected independently from the group consisting of H, HO—, CH₃O—, CH₃—, HOCH₂CH₂—, HOCH₂CH₂OCH₂CH₂—, NH₂CH₂CH₂—, R⁷NHCH₂CH₂—, (R⁷)₂NCH₂CH₂—, NH₂CH₂CH₂NHCH₂CH₂—, R⁷NHCH₂CH₂NHCH₂CH₂—, (R⁷)₂NCH₂CH₂NHCH₂CH₂—, R⁸CO—, or a mono-, di-, or tri-cyclic aryl from C₆ to C₁₄;
and wherein R⁷ is an alkyl from C₁ to C₅; and R⁸ is H₂N—, HOHN—, alkyl from C₁ to C₁₀, alkenyl from C₂ to C₁₀, or phenyl.

41. A molecule of claim 1 wherein R¹ is H, R³ is H, R⁴ is H, R⁵ is H, R⁶ is C₆H₆, U is H, Z is H, W and Y are —OC(CH₃)₂O—, X¹ is O, X² is O, and R² is selected independently from the group consisting of H, HO—, CH₃O—, CH₃—, HOCH₂CH₂—, HOCH₂CH₂OCH₂CH₂—, NH₂CH₂CH₂—, R⁷NHCH₂CH₂—, (R⁷)₂NCH₂CH₂—, NH₂CH₂CH₂NHCH₂CH₂—, R⁷NHCH₂CH₂NHCH₂CH₂—, (R⁷)₂NCH₂CH₂NHCH₂CH₂—, R⁸CO—, or a mono-, di-, or tri-cyclic aryl from C₆ to C₁₄;
and wherein R⁷ is an alkyl from C₁ to C₅; and R⁸ is H₂N—, HOHN—, alkyl from C₁ to C₁₀, alkenyl from C₂ to C₁₀, or phenyl.

42. A molecule having the structure 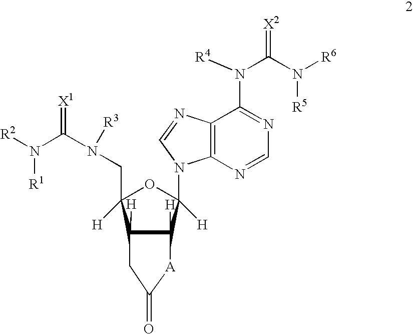
wherein:
R¹, R², R⁵, and R⁶, are members selected independently from the group consisting of H, HO—, CH₃O—, CH₃—, HOCH₂CH₂—, HOCH₂CH₂OCH₂CH₂—, NH₂CH₂CH₂—, R⁷NHCH₂CH₂—, (R⁷)₂NCH₂CH₂—, NH₂CH₂CH₂NHCH₂CH₂—, R⁷NHCH₂CH₂NHCH₂CH₂—, (R⁷)₂NCH₂CH₂NHCH₂CH₂—, R⁸CO—, a mono-, di-, or tri-cyclic aryl from C₆ to C₁₄, a mono-, di-, or tri-cyclic aryl from C₆ to C₁₄ mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂;
and wherein R⁷ is an alkyl from C₁ to C₅; R⁸ is H₂N—, HOHN—, alkyl from C₁ to C₁₀, alkenyl from C₂ to C₁₀, or phenyl; and R⁹ is alkyl from C₁ to C₁₂;
R³, R⁴, are members selected independently from the group consisting of H, HO—, CH₃—, or CH₃CH₂—;
X¹ and X² are members selected independently from the group consisting of O and S;
A is a member selected from the group consisting of O, and NR¹⁰;
and wherein le is a member selected independently from the group consisting of H, HO—, CH₃—, or CH₃CH₂—.

43. A molecule of claim 42 wherein R¹ is H, R² is CH₃, R³ is H, R⁴ is H, R⁵ is H, X¹ is O, X² is O, A is O, and R⁶ is a member selected independently from the group consisting of a mono-, di-, or tri-cyclic aryl from C₆ to C₁₄.

44. A molecule of claim 43 wherein R⁶ is a member selected independently from the group consisting of a mono-, di-, or tri-cyclic aryl from C₆ to C₁₄ mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂;
and wherein R⁹ is alkyl from C₁ to C₁₂.

45. A molecule of claim 43 wherein R⁶ is phenyl.

46. A molecule of claim 42 wherein R¹ is H, R² is CH₃, R³ is H, R⁴ is H, R⁵ is H, X¹ is O, X² is O, A is NH, and R⁶ is a member selected independently from the group consisting of a mono-, di-, or tri-cyclic aryl from C₆ to C₁₄.

47. A molecule of claim 46 wherein R⁶ is a member selected independently from the group consisting of a mono-, di-, or tri-cyclic aryl from C₆ to C₁₄ mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂; an O, N, or S mono- or bi-cyclic heterocycle having from two to nine carbon atoms, and an O, N, or S mono- or bi-cyclic heterocycle having from two to nine carbon atoms and mono-, di-, tri-, or poly-substituted with a member selected independently from the group consisting of F, Cl, Br, I, alkoxy (R⁹O—), nitro (NO₂), nitroso (NO), azido (N₃), alkyl from C₂ to C₁₂, alkenyl from C₂ to C₁₂, alkynyl from C₂ to C₁₂, or acyl from C₂ to C₁₂;
and wherein R⁹ is alkyl from C₁ to C₁₂.

48. A molecule of claim 46 wherein R⁶ is phenyl.
